(19) United States
(12) Patent Application Publication
(10) Pub. No.: US 2007/0226175 A1
(43) Pub. Date: Sep. 27, 2007

(54) Automated medical safety monitoring systems and methods

(75) Inventors: Frederic Scott Resnic, Needham, MA (US); Richard Cope, Dracut, MA (US); Michael E. Matheny, Brookline, MA (US); Barry Coflan, North Andover, MA (US)

Correspondence Address: WOLF GREENFIELD & SACKS, P.C., 600 ATLANTIC AVENUE, BOSTON, MA, 02210-2206, US

(73) Assignees: The Brigham and Women's Hospital, Inc., Boston, MA (US); Coping Systems, Inc., Andover, MA (US)

(21) Appl. No.: 11/715,609

(22) Filed: Mar. 8, 2007

Related U.S. Application Data
(60) Provisional application No. 60/780,437, filed on Mar. 8, 2006.

(51) Int. Cl. G06F 17/30; A61K 9/22; G06F 7/00

(52) U.S. Cl. 707/1; 604/890.1

(57) ABSTRACT

In one aspect, a medical surveillance system for monitoring the safety of at least one medical entity is provided. The medical surveillance system comprises at least one database to store information related to the at least one medical entity, the at least one database being periodically updated with current information about the at least one medical entity, and at least one controller coupled to the database, the at least one controller configured to perform at least one statistical operation on at least a portion of the information stored in the at least one database, the at least one statistical operation being performed periodically so as to generate at least one continuous outcome, the controller configured to trigger an alert when the at least one continuous outcome indicates the occurrence of at least one adverse event associated with the at least one medical entity.

### RELATED APPLICATION

This application claims priority under 35 U.S.C. § 119(e) to U.S. Provisional Application Ser. No. 60/780,437, entitled “AUTOMATED MEDICAL SAFETY MONITORING SYSTEMS AND METHODS,” filed on Mar. 8, 2006, which is herein incorporated by reference in its entirety.

### FIELD OF INVENTION

The present invention relates to medical safety monitoring of adverse events related to medical devices, drugs, medical procedures, etc.

### BACKGROUND

In general, medical devices, drugs and/or medical practices and procedures (hereinafter medical entities) are regulated by the Food and Drug Administration (FDA). New medical entities typically must be approved by the FDA before entering the market for public use. The FDA performs various tests and studies on medical entities in an effort to ascertain whether the medical entity is safe for use. For example, the FDA may perform various tests on a new medical device (e.g., drug eluding stents, cardio-defibrillators, vascular closure devices, etc.) to demonstrate the medical device efficacy prior to approving the device for marketing. Similarly, a new drug typically must pass a battery of tests before it is approved to be marketed to the general public.

While pre-approval randomized clinical trials required for demonstrating medical device efficacy have important safety endpoints, it is generally observed that such trials may be inadequate to comprehensively assess the safety of new medical devices. The inability of randomized trials to ensure safety for the population at large are due in part to the preferential inclusion of highly selected patient populations (as opposed to the application of devices to populations outside of those studied in the trials, which commonly occurs after a device is approved and in the marketplace), the very low frequency of adverse events, and the rapid dissemination of the new technology to practitioners who may not be as expert in the device use or in patient selection as those involved in the trials.

The FDA currently relies on a collection of passive reporting systems for collecting information regarding medical device failures following approval for use. These systems, such as the medical device reporting (MDR) system, the MedSun system, MedWatch and the “Adverse Event Reporting System” rely on user initiated submission of adverse events to vendors, and mandatory submission of this information to the FDA. These reporting systems are plagued by their inability to assess accurate event rates due to under-reporting and an absence of mechanisms to collect accurate overall usage statistics. The U.S. General Accounting Office has estimated that less than one percent of adverse events involving medical devices are reported to the FDA. In addition, the FDA has limited access to the number of patients exposed to new devices, and often can make no accurate inferences regarding the true rates of adverse events.

Recent examples of problems associated with assessing post-market medical device safety have involved two FDA approved drug eluting coronary stent systems: Cypher™ (Cordis Corp., NJ) and Taxus™ (Boston Scientific Corp., Natick, Mass.). Cypher was approved for market release in April 2003. By December 2003, the FDA had received reports of over 300 cases of subacute stent thrombosis (SAT), a dangerous complication of stent implantation that typically leads to heart attack or, in some instances, death. These reports raised serious concerns regarding the safety of the device. Compounding this problem was the exponential growth in the use of the device in the U.S., with over 200,000 devices implanted per quarter by the beginning of 2004. Unfortunately, given the tools available for safety surveillance, the FDA lacked detailed clinical data on the types of patients who were receiving the devices as well as accurate information regarding the overall number of devices implanted.

In October 2003, the FDA issued a public health notification about the Cypher stent to remind clinicians to follow the labeled instructions for use for the device. After further review of post-market surveillance registries as well as the release of further randomized trial data, the FDA concluded that there was no increase in the risk of SAT over bare metal stents. Therefore, the FDA made a second notification to all U.S. cardiologists concluding that the Cypher Stent was safe.

In addition to the Cypher drug eluting stent SAT concern, the second drug eluting coronary stent approved for use in the U.S., the Taxus stent, was subject to three separate manufacturer recalls within 6 months of release for failures of balloon deflation, which were identified in 40 patients and associated with 18 serious injuries, including one death. Additionally, the very large and costly recalls of implantable cardio-defibrillators in 2005 have highlighted the deficiency in conventional safety surveillance programs.

### BRIEF DESCRIPTION OF THE DRAWINGS

FIG. 1 illustrates various sources of data that may be used to facilitate a medical safety monitoring system, in accordance with some embodiments of the present invention;

FIG. 2 illustrates a medical safety monitoring system, in accordance with some embodiments of the present invention;

FIG. 3 illustrates a safety monitoring system, in accordance with some embodiments of the present invention;

FIG. 4 illustrates various analytic methods that may be performed by a safety monitoring system, in accordance with some embodiments of the present invention;

FIG. 5 illustrates results from performing a periodic (e.g., monthly) SPC uniform expectation analysis on safety information, in accordance with some embodiments of the present invention;

FIG. 6 illustrates a report indicating all alerts over a given interval, in accordance with some embodiments of the present invention;

FIG. 7 illustrates an example of visualizing data in a report, in accordance with some embodiments of the present invention;

FIG. 8 illustrates results from performing a cumulative SPC uniform expectation analysis on safety information over the course of an 18 month interval, in accordance with some embodiments of the present invention;

FIG. 9 illustrates various statistical methods that may be used in a safety monitoring system, in accordance with some embodiments of the present invention;

FIG. 10 illustrates a covariate scoring for an LR model for determining the probability of subacute stent thrombosis (SAT) given the listed factors, in accordance with some embodiments of the present invention;

FIG. 11 illustrates results of an LR analysis of cases of SAT resulting from the use of a DES device, in accordance with some embodiments of the present invention;

FIG. 12 illustrates priors established for an initial prior probability density function (PDF) using a method defining alert thresholds by minimum overlap in prior and posterior PDF's, in accordance with some embodiments of the present invention;

FIG. 13 illustrates the evolution of the PDF's for the high risk strata defined in FIG. 12, in accordance with some embodiments of the present invention;

FIG. 14 illustrates a screen shot of an introductory page of a browser based interface provided to the operator to interact with the safety monitoring system, in accordance with some embodiments of the present invention;

FIG. 15 illustrates the interface provided to establish and customize one or more analyses of the data, in accordance with some embodiments of the present invention;

FIG. 16 illustrates a screen shot of a report of one analysis, in accordance with some embodiments of the present invention;

FIG. 17 illustrates the instantiation of an LR analysis on the vascular closure device, in accordance with some embodiments of the present invention;

FIG. 18 illustrates the screen shot for establishing weightings for desired beta factors in a linear regression (LR) analysis, in accordance with some embodiments of the present invention;

FIG. 19 illustrates an alert structure set up for the LR analysis, in accordance with some embodiments of the present invention;

FIG. 20 illustrates the page generated when the “Result” button is selected, in accordance with some embodiments of the present invention;

FIG. 21 illustrates a “Summary per Period” report, accordance with some embodiments of the present invention;

FIG. 22 illustrates a “Cumulative Expected with Cumulative Observed” report, in accordance with some embodiments of the present invention;

FIG. 23 illustrates alerts that have been defined for an LR analysis, in accordance with some embodiments of the present invention;

FIG. 24 illustrates an alert structure established for a statistic process control (SPC) analysis, accordance with some embodiments of the present invention;

FIG. 25 illustrates alerts that were generated from the alert structure in FIG. 24 for all the periods over an 18 months interval, in accordance with some embodiments of the present invention;

FIG. 26 illustrates results showing the cumulative observed data versus the expectation and confidence intervals for a low risk strata, in accordance with some embodiments of the present invention;

FIG. 27 illustrates results showing the cumulative observed data versus the expectation and confidence intervals for a high risk strata, in accordance with some embodiments of the present invention;

FIG. 28 illustrates the results page for an established Bayesian Update System (BUS) analysis, having a low risk and high risk strata, in accordance with some embodiments of the present invention;

FIG. 29 illustrates an alert defined for the analysis of FIG. 28, in accordance with some embodiments of the present invention;

FIG. 30 illustrates the report of cumulative observed data versus the expected for a low risk strata of the BUS analysis, in accordance with some embodiments of the present invention;

FIG. 31 illustrates the report of cumulative observed data versus the expected for the high risk strata of the BUS analysis, in accordance with some embodiments of the present invention; and

FIG. 32 illustrates the cumulative PDF's evolution for the low risk strata over a 3 year interval, in accordance with some embodiments of the present invention.

### DETAILED DESCRIPTION

As discussed above, pre-approval testing of medical entities (e.g., medical devices, drugs, medical procedures, etc.) may fail to prevent marketing of unacceptably unsafe and/or potentially dangerous medical entities. Post-market safety monitoring, therefore, may be an important feature of ensuring that such medical entities are generally safe (e.g., involve acceptable risks) and/or are not defective. However, there is no current methodology for implementing such a monitoring system. Moreover, there are relatively significant challenges involved in developing a system for effective post-market monitoring.

Rapid dissemination of new medical technology, lack of standards in data collection, and lack of information integration, at least in part, have frustrated development of anything beyond relatively crude and passive safety monitoring tools. For example, conventional post-market safety monitoring, if performed at all, has been achieved by analyzing historic data that has been compiled over a number of years to determine, for example, if an unsatisfactory rate of adverse events have occurred. That is, conventional monitoring, when performed at all, has been done retrospectively. Moreover, the only available historic data available even for retrospective analysis is often a local compilation of information, for example, obtained within a given hospital, and therefore may not be representative of events on a larger scale. Accordingly, it may be difficult to accurately identify the source of a problem or to correctly identify a problem at all. For example, it may be difficult to assess whether an identified problem resulted from improper use of a device, a defect in the device itself, an unknown side effect of device implementation, or some combination thereof.

In addition, conventional solutions are insufficient, in part, because even if a problem is identified, by the time a conventional retrospective analysis is performed on data obtained over a period of time, significant injury may have already resulted, including perhaps numerous serious injuries and/or deaths. For example, an analysis performed on post-market information collected over several years that identifies a defective device, or high rate of complications resulting from the use of the device may have already resulted in a significant number of injuries and/or deaths by the time the analysis is performed and one or more adverse events identified. Applicant has appreciated that by continuously analyzing post-market data, problems associated with various medical entities may be identified earlier and as they arise, rather than retrospectively after significant harm may have already occurred. One embodiment according to the present invention includes a medical safety monitoring system that performs substantially continuous analysis of safety information and generates generally real-time alerts of adverse events associated with one or more monitored medical entities.

The term “continuous” as it relates to analysis and/or monitoring refers herein to performing the associated acts generally contemporaneously with the time data or information becomes available. This is in contrast to conventional systems that perform retrospective analysis on historical data that has been compiled for some discrete amount of time. For example, a continuous monitoring system may perform one or more statistical analyses each time safety information is added to a database included as part of a monitoring system. The update of one or more databases, for example, may trigger the monitoring system to re-analyze the data in view of the new information to report on adverse events and/or determine whether one or more alerts should be generated.

Some embodiments according to the present invention include a medical surveillance system for monitoring the safety of at least one medical entity, the medical surveillance system comprising at least one database to store information related to the at least one medical entity, the at least one database being periodically updated with current information about the at least one medical entity, and at least one controller coupled to the database, the at least one controller configured to perform at least one statistical operation on at least a portion of the information stored in the at least one database, the at least one statistical operation being performed at least each time the at least one database is updated with the current information so as to generate at least one continuous outcome, the controller configured to trigger an alert when the at least one continuous outcome indicates the occurrence of at least one adverse event associated with the at least one medical entity.

Some embodiments according to the present invention include a method of monitoring the safety of at least one medical entity, the method comprising acts of obtaining periodically updated information relating to the safety of the at least one medical entity, performing, each time updated information is obtained, at least one statistical operation on at least a portion of the information to generate at least one continuous outcome, and generating an alert when the at least one continuous outcome indicates the occurrence of at least one adverse event associated with the at least one medical entity.

Some embodiments according to the present invention include a computer readable medium encoded with a program for execution on at least one processor, the program, when executed on the at least one processor, performing a method of monitoring the safety of at least one medical entity, the method comprising acts of obtaining periodically updated information relating to the safety of the at least one medical entity, performing, each time updated information is obtained, at least one statistical operation on at least a portion of the information to generate at least one continuous outcome, and generating an alert when the at least one continuous outcome indicates the occurrence of at least one adverse event associated with the at least one medical entity.

One impediment to the development of a medical safety monitoring system is the lack of available information. While safety information may be routinely collected, the information is often derived from different sources, and may only be reflective of a particular locality such a single hospital or relatively local region. To compound the problem, data collection is not integrated and there generally are no standards for format, the type of information collected and/or consistency in data collection. As discussed above, when information is available at all, it is typically compiled locally, for example, within a particular hospital and therefore is only indicative of activity that occurred within the hospital, thus resulting in data that is relatively sparse. Moreover, once this information is analyzed, the results may be published, but the data is typically not available outside of the hospital or organization responsible for collecting the data. Accordingly, the lack of available and/or satisfactory data sources has contributed to the failure of the concept of a continuous and automatic medical safety monitoring system from being envisioned and/or developed.

In recent years, new possible sources of safety information have arisen due, in part, to expanded use of voluntary post-market safety reporting, and due, in part, to recent government mandated post-market reporting. For example, some states have enacted regulations that require institutions to report information related to the use of various medical entities, such as medical devices, drugs, etc. In some instances, as a condition of using a particular medical entity, the FDA may require an institution to implement certain reporting mechanisms to carry out what is referred to as a post-approval surveillance study (PAS). Applicant has appreciated that newly available information sources may be used to facilitate a post-market medical safety monitoring system.

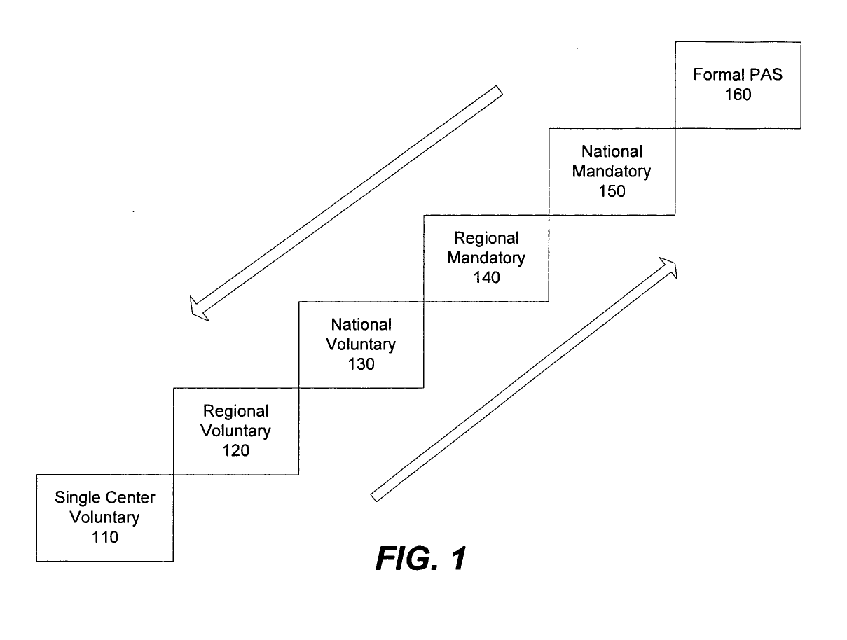

FIG. 1 illustrates various sources of data that may be used to facilitate a medical safety monitoring system. In general, the cost and reliability of the information is lowest at the bottom left of the diagram and increases generally in cost and reliability in directions up and to the right, as indicated by the arrows. A first source of data may be single center voluntary information 110. The single center voluntary database describes a repository of information collected by a single institution, for example, a hospital, clinic or medical center. Many medical centers have collected such information for a relatively long time, for example, as part of institutional protocols established at the individual medical facilities. However, since the collected information is local in nature, the results may not be reflective of actual medical entity safety on a larger scale.

Regional voluntary database 120 refers generally to collaborations between a plurality of medical centers, typically in a particular geographic area, and/or industry coordinated clinical registries for a particular region or area of medicine. One example of a regional voluntary database is the NNE Cooperative. The regional voluntary information may be more reflective of actual safety, but is generally more expensive to collect, coordinate and evaluate. National voluntary database 130 may include voluntary registries such as the American College of Cardiology—National Cardiovascular Data Repository (ACC-NCDR), national prospective registries such as the National Heart, Lung & Blood Institute (NHLBI) Dynamic Registry, etc. As should be appreciated, national voluntary databases are more reflective of information on a wider scale, but may still suffer from reliability issues due, in part, to the voluntary nature of the data collection.

For example, in the Cypher™ stent case, Cypher™ was approved for market release in April 2003. As discussed above, by December 2003, the FDA had received reports of over 300 cases of SAT. As a result, in October 2003, the FDA issued a public health notification about the Cypher™ stent. After further review of detailed post-market surveillance registries as well as the release of further randomized trial data, the FDA concluded that there was no increase in the risk of SAT over bare metal stents. Therefore, the FDA made a second notification to all U.S. cardiologists concluding that the Cypher™ Stent was safe.

The notoriety of the events, coupled with the explosive growth in the use of drug-eluting stents in the U.S., led to a dramatic increase in the number of adverse events reported regarding Cypher™ stents after the first FDA notification. Upon review of the publicly available adverse event reports for the Cypher™ stents, a comparison was made of the number of device-associated events with the number of units sold during each quarter. The results showed that there was a dramatic increase in the number of reports per unit sold in each quarter following the initial FDA notification until the FDA's second, more reassuring notification, after which the adverse event rates dropped precipitously. This bias toward “stimulated reporting” highlights the difficulties in interpreting the frequency of events reported through voluntary registries.

Regional mandatory database 140 may include registries compiled as a result of, for example, state regulations mandating the reporting of medical entity information. At least New York, Pennsylvania, New Jersey and Massachusetts have passed regulations mandating medical entity reporting. Other regional mandatory databases include the Centers for Medicare and Medicaid Services (CMS) mandated registry, for example, that mandates reporting of carotid stents, ICDs, etc. In general, mandated registries yield higher reliability data due, in part, to the fact that all users of a medical entity must produce reports, rather than a selection of those that choose to voluntary provide the reporting information, thus avoiding the associated bias. No current national mandatory registries are in place, but federal regulation may change this in the future.

PAS repositories 160 are also a possible source of medical information. As discussed above, the FDA often requires a user (e.g., a hospital, clinic, medical center, etc.) of a particular medical entity to provide detailed reporting on the medical entity as a condition of using the device. PAS reporting provides the FDA with post-market data and may allow a medical entity to be marketed sooner, thus effectively balancing the public health and safety risks with the benefit of access to new medical entities. It should be appreciated that the above databases are merely examples of data sources that may be utilized to obtain information that can used by a medical safety monitoring system. However, any source of medical information may be used, as the aspects of the invention are not limited in this respect.

In one embodiment according to the present invention, a medical safety monitoring system is adapted to communicate with one or more databases to provide generally real-time assessment of the safety of monitored medical entities. The medical safety monitoring system may then exploit the data obtained from a variety of sources and perform one or more continuous statistical analyses on the data, as described in further detail below.

Following below are more detailed descriptions of various concepts related to, and embodiments of, methods and apparatus according to the present invention. It should be appreciated that various aspects of the invention described herein may be implemented in any of numerous ways. Examples of specific implementations are provided herein for illustrative purposes only. In addition, the various aspects of the invention described in the embodiments below may be used alone or in any combination, and are not limited to the combinations explicitly described herein.

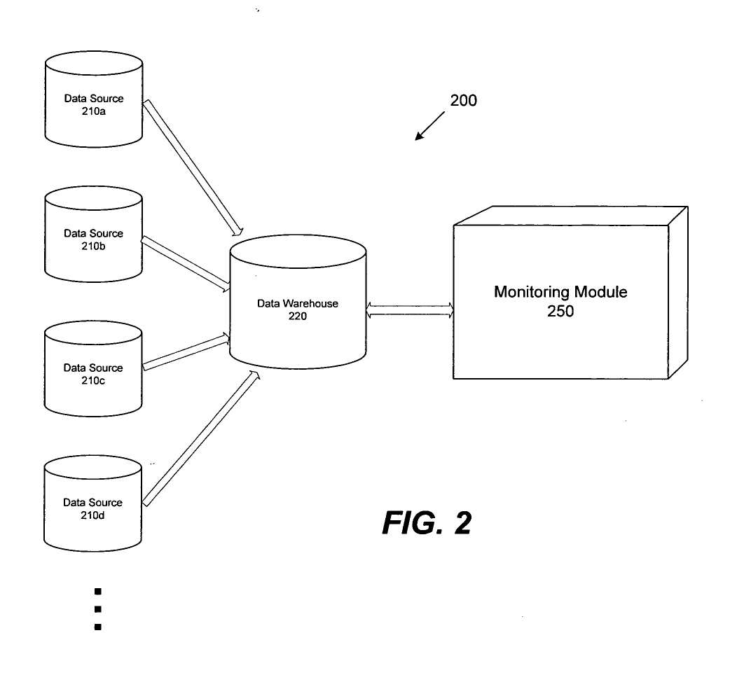

FIG. 2 illustrates a medical safety monitoring system, in accordance with some embodiments of the present invention. Monitoring system 200 includes a plurality of data sources 210 (e.g., data sources 210a-210d) for storing medical safety information. Data sources 200 may be of any type and compiled by whatever means. For example, the data sources may include one or any combination of voluntary reporting databases, mandatory reporting databases, post-approval surveillance studies, etc. It should be appreciated that any number of data sources may be used, which may be of any type, as the aspects of the invention are not limited in this respect.

Monitoring system 200 may also include a data warehouse 220. The data warehouse may be coupled to each of the plurality of data sources to provide a centralized repository of reporting information. The data warehouse may facilitate the collection of reporting information from a variety of different sources, formats and reporting mechanisms and convert them into a desired format to be analyzed (e.g., a flat file format). Accordingly, data warehouse 220 provides an abstraction layer between the data source and monitoring module 250. Data warehouse 220, for example, may include an on-line analytical processing (OLAP) design to facilitate relatively quick answers to analytical queries that are, for example, dimensional in nature. However, data warehouse 220 may be of any design, for example, on-line transaction processing (OLTP), or any multi-dimensional, navigational, hierarchical and/or relational database, etc., as the aspects of the invention are not limited in this respect.

Data warehouse 220 permits new data sources of any type to be added to the system without having to reconfigure and reprogram the monitoring module to interface with the data source. That is, the number, type and nature of the data sources may be invisible to monitoring module 250. It should be appreciated that data warehouse 220 is not required and may be eliminated according to some embodiments. For example, monitoring module 250 may be directly coupled to one or more data sources and configured to receive reporting data directly from the data source(s).

Monitoring module 250 includes one or more programs adapted to continuously monitor the data collected from data sources 110. In particular, when new data is entered into any of the data sources, monitoring module 250 may perform an analysis on the new data independently or in connection with previously entered data. For example, monitoring module 250 may be adapted to perform any number and type of programmed analytical queries to data warehouse 220 to accomplish a desired set of one or more monitoring or surveillance tasks, as described in further detail below.

Monitoring module 250 may be configured to generate an alert when the analysis suggests that an adverse event has occurred. The term “adverse event” refers herein to any numerical or statistical result that falls outside of a set threshold value, interval, level or expectation, etc. For example, an adverse event may be a reported number of deaths or complications that exceeds a predetermined expectation for the rate of such an event. An adverse event may be a trend (e.g., a velocity) in the rate of a particular occurrence that increases more rapidly than expected. An adverse event may be any occurrence or circumstance for which monitoring is desired.

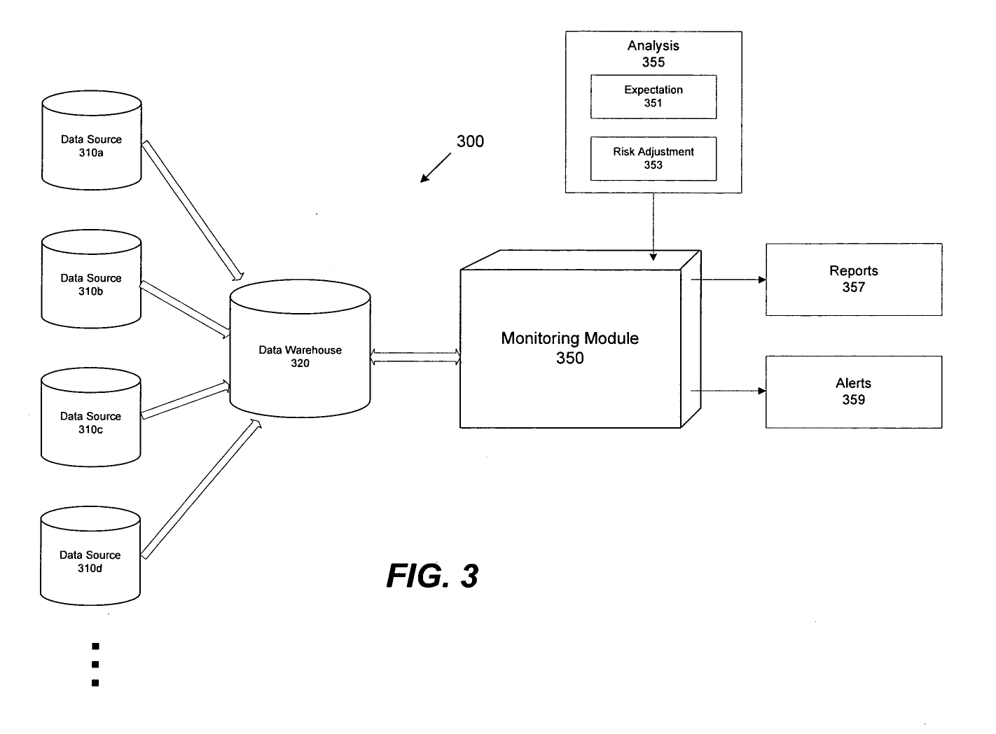

FIG. 3 illustrates a safety monitoring system, in accordance with some embodiments of the present invention. Safety monitoring system 300 may be similar in some respects to safety monitoring system 200 illustrated in FIG. 2. In FIG. 3, possible inputs and outputs of the safety monitoring system are illustrated. In particular, monitoring module 350 may perform one or more statistical analyses on information stored in data warehouse 320. For example, monitoring module 350 may perform one or more of Bayesian analysis, statistic process control (SPC) and logistic regression (LR) on the data by appropriately querying the information, as described in further detail below. When one or more of the statistical analyses indicates the occurrence of an adverse event, monitoring module 350 may generate an alert 359. Some exemplary alerts are discussed in further detail below.

In addition, the result of a particular analysis may be output as one or more reports 357. For example, results of an analysis may be graphed as a function of time, so that a user can view trends or otherwise visualize the results of the analysis. For example, an operator may examine the data in a report to determine whether additional action is necessary. Reports 357 may be of any form and contain any information, as the aspects of the invention are not limited in this respect. Some exemplary reports are discussed in further detail below.

In order for monitoring system 350 to generate reports and/or alerts, the monitoring system may be programmed to perform one or more analyses of the data (e.g., perform one or more analytical queries or series of queries to data warehouse 320). In addition, to generate an alert, monitoring system 350 may need to be instructed as to what amounts to an adverse event and what amounts to a so-called expected event. That is, monitoring system 350 may need to be programmed to understand what results or set of results justify an alert. For example, one statistic that may be important to safety monitoring is the ratio of the number of identified events to the number of patients using the medical entity. For example, in the context of drug eluding stents (DES), the number of patients who developed SAT complications to the number of patients with implanted stents may be a valuable measure in assessing the safety of the drug eluding stent. If the ratio should exceed some expected value, an alert may be raised to indicate the occurrence of an adverse event.

However, it is not always straight forward to determine what expectations should be. Some devices and/or procedures are inherently riskier than others. Similarly, complications, side-effects, effects of device failure, etc. may involve widely varying levels of seriousness; from relatively mild complications to serious injury or death. Accordingly, it may be difficult to properly assess what level of event rates should be tolerated, and which exceed the expectations for a particular device, drug, procedure, etc.

Moreover, there are many variables that can result in failure of the medical entity. For example, for a medical device, the event (e.g., complication, injury, death, etc.) may have resulted from one or any combination of device failure, improper use (user defect), improper implantation or installment (physician or operator defect), use outside or beyond the indicated use, etc. To further complicate matters, acceptable rates of events may change over time. For example, event rates may be expected to be higher at times nearer to the medical entity's appearance on the market. Improvements in technique and familiarity with a device, for example, may reduce the rate of events. In addition, improvements in the device may also tend to reduce the rate of events. Such factors are relatively difficult to account for.

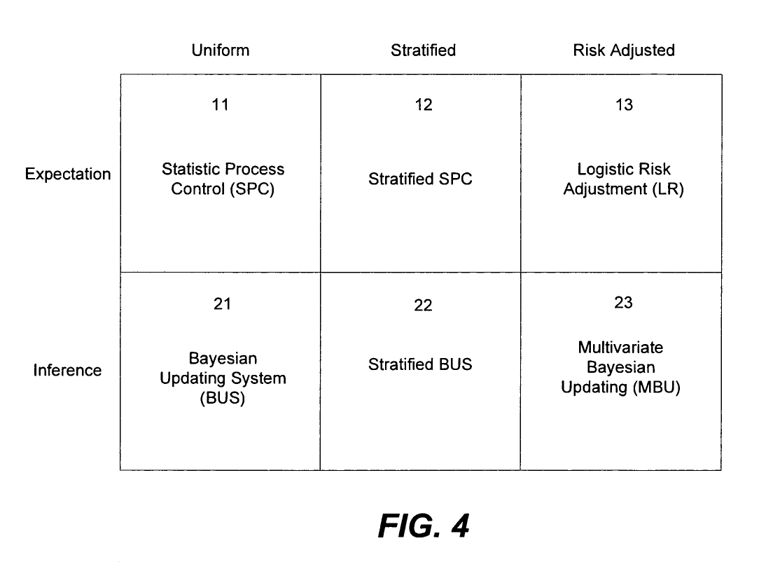

FIG. 4 illustrates various analytic methods that may be performed by a safety monitoring system, in accordance with some embodiments of the present invention. As a general overview of FIG. 4, the first row illustrates various expectation methods, and the second row illustrates various inference methods. The first column illustrates the respective methods performed uniformly on the data. That is, the data is analyzed as a whole, generally without any further separation of the data and/or consideration of the source of the data or criteria therein.

The second column illustrates the respective methods using risk stratification. Risk stratification is a process by which a given sample is subdivided into discrete groups based on predefined criteria, creating separation in the data to allow concurrent and potentially different analyses to be performed on each subset. As discussed in further detail below, risk stratification may facilitate the discovery of patterns or unacceptable event rates for particular classes or strata, rather than the entire population. Risk stratification may facilitate generating alerts for a particular strata before corresponding alerts would have been generated if the data were considered as a whole, that is, considered uniformly as shown in the first column.

The third column illustrates the respective methods using risk adjustment. Risk adjustment performs analysis with expectations that change over time. That is, levels of event rates that are considered expected and levels that are considered adverse may change over time, for example, as function of the received data. In particular, threshold values, expectation intervals, etc., may be automatically adjusted based on the data accumulated at a given point in time. In this way, measures that trigger alerts may be adjusted to more adequately reflect true adverse events by considering the data upon which those alerts operate.

The various analysis methods may be used alone, or in any combination to implement a medical safety monitoring system. Uniform, risk stratified and risk adjusted methods are described below in connection with various statistical operations including Bayesian analysis, statistical process control (SPC), and logistic regression. However, it should be appreciated that other statistical processes and/or analysis may be used to facilitate generally continuous analysis of a medical entity, as the aspects of the invention are not limited in this respect.

In method 11 (i.e., row 1, column 1), an exemplary uniform expectation statistical method may be used. In particular, statistical process control (SPC), often used to identify product defect rates in manufacturing processes may be used to generate alerts and/or reports concerning the safety of one or more medical entities. SPC may be used to compare observed adverse event rates to alerting boundaries set according to expectations. For example, there may be an expectation of a certain number of adverse events in patients getting a certain medication, or the number of failures of a certain procedure and/or complications arising from use of a particular medical device.

Safety expectations may be derived from previously published trial data, data on similar devices/drugs/procedures, clinical trial data from pre-market testing or other observed empirical data, and/or via solicitation from one or more experts. The various expectation values obtained may be weighted in different ways, or used independently to run separate expectation analyses. Once one or more expectations are set, corresponding SPC analysis may be performed on the data. When event rates exceed the one more expectations, an alert may be generated to indicate the occurrence of an adverse event. Analysis may be performed on the data viewed over a particular time interval (i.e., periodically), or viewed over the entire span of the data, (i.e., cumulatively) as discussed in further detail below.

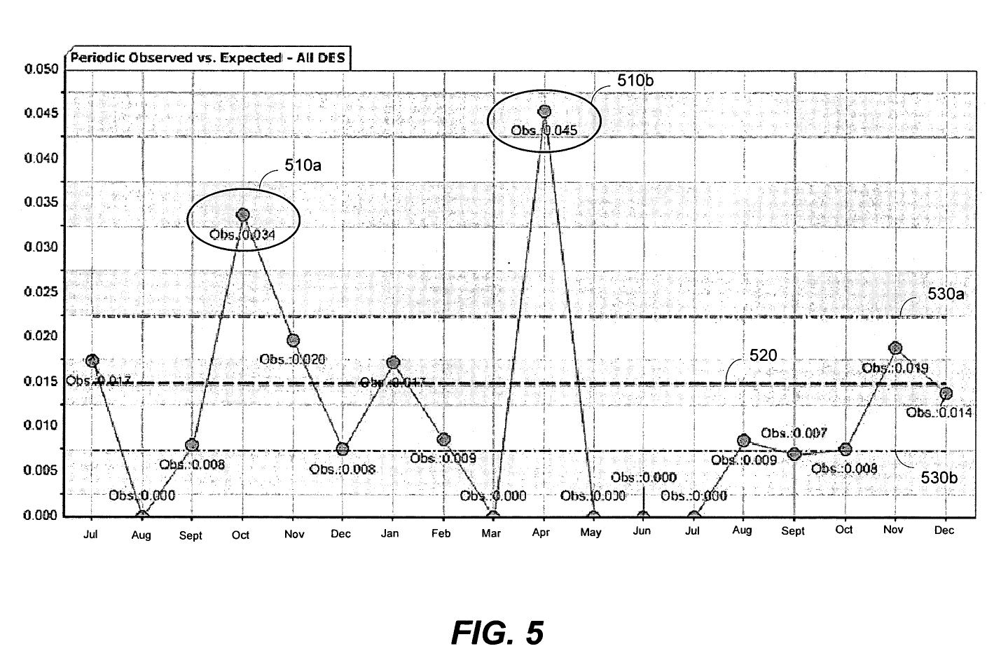

FIG. 5 illustrates results from performing a periodic (e.g., monthly) SPC uniform expectation analysis on safety information, in accordance with some embodiments of the present invention. That is, all the data for a prescribed interval (in this instance, a month) of time is analyzed independent of other time intervals. In FIG. 5, the underlying data relates to deaths resulting from the implantation of a drug eluding stent (DES). That is, the x-axis shows the percentage of deaths (i.e., the total number of stent implantations divided by the number of deaths) resulting from the drug eluding stent. It should be appreciated that the safety information may relate to any type of event corresponding to any medical entity, and the use of a drug eluting stent database is merely exemplary. Moreover, a periodic analysis may be performed over any desired interval (e.g., a day, week, month, year, etc.), as the aspects of the invention are not limited in this respect.

The SPC analysis, the results of which are illustrated in FIG. 5, was instantiated with an expectation 520 (i.e., 0.015), which may have been derived from one or any combination of sources described above (e.g., previous empirical data, published results, expert opinion, etc.). The analysis uses a 95% confidence interval (CI) 530 having upper bound 530a (i.e., 0.0225) and lower bound 530b (i.e., 0.0075) as the threshold to determine when the event rate has exceeded the expectation. That is, the confidence interval forms the limits on acceptable event rates, outside of which the event rates may be considered adverse. The 95% percent confidence interval is often used as a threshold of statistical improbability to establish when a true deviation from the expectation has occurred. However, any confidence interval, or threshold derived by any means may be used to distinguish acceptable or expected events from adverse events, as the aspects of the invention are not limited in this respect.

The SPC analysis illustrated in FIG. 5 was configured to generate an alert whenever the analysis resulted in a number that exceeded confidence interval 520. As shown, alerts were generated for the months of October and April. It should be appreciated that the alerts may be generated in any form. For example, an alert may cause a pop-up window to be created to notify an operator of the alert, or an icon may change colors, or the appearance of a graphic user interface of the monitoring system can otherwise change to indicate that an alert has been generated. An alert may create and send an email to one or more email accounts notifying the recipient that an alert has been generated. A telephone call or text message may additionally be generated to notify one or more operators that an alert has been generated. Any type of alert may be used, as the aspects of the invention are not limited in this respect.

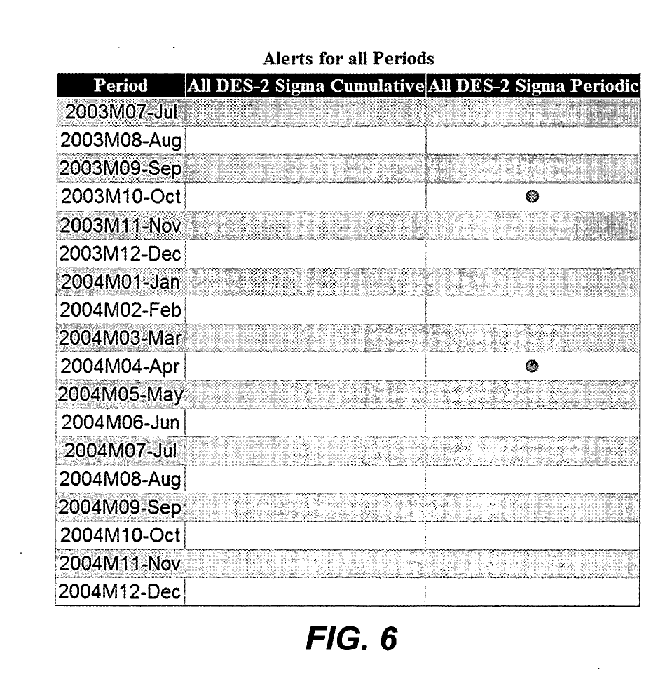

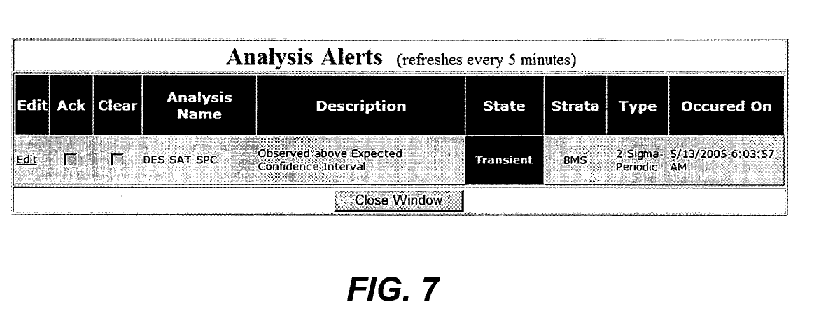

FIG. 5 also illustrates one example of a report (e.g., a visualization of the data and/or analysis) that may be generated by the monitoring module (e.g., monitoring module 350). The report may be generated at any time, for example, on a monthly basis or upon instruction from an operator to generate the report. FIGS. 6 and 7 illustrate alternative methods of visualizing the data to be reported. Any type of report may be generated that formats the data in any fashion desired, as the aspects of the invention are not limited in this respect.

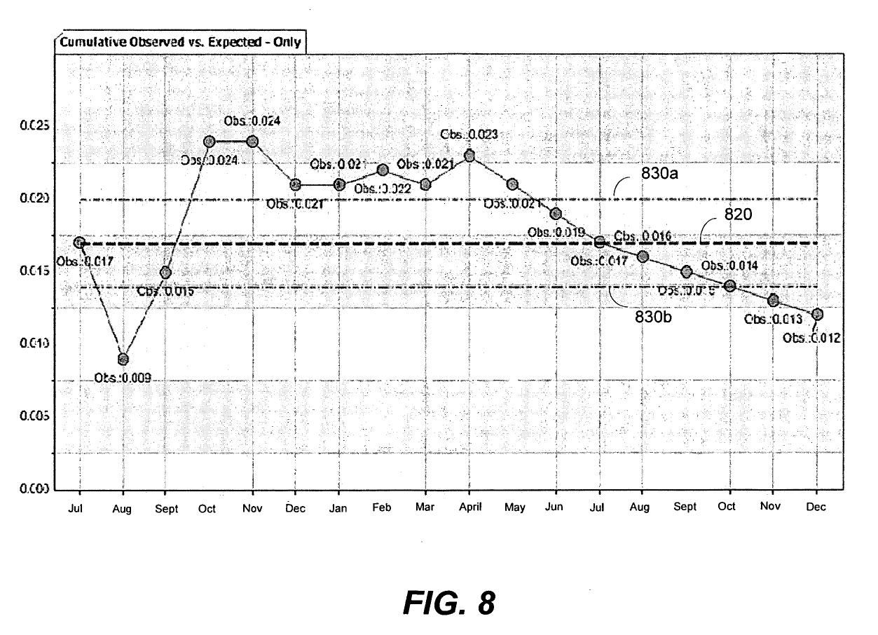

FIG. 8 illustrates results from performing a cumulative SPC uniform expectation analysis on safety information over the course of an 18 month interval. Cumulative methods perform an analysis on all of the data received at the moment of the analysis. In FIG. 8, a 95% percent confidence interval 830 (i.e., ranging between upper bound 830a and lower bound 830b) about expectation 820 (i.e., of approximately 0.017) is used to generate alerts. Since the analysis is cumulative, the results illustrated for each month is the DES death percentage over the entire interval up until the corresponding month, rather than just for the corresponding month as in the periodic analysis illustrated in FIG. 5. As shown, alerts were generated from October-May.

It is interesting to note that the cumulative report shows high percentage rates at the beginning of the interval and a steady decrease after May. The cumulative report highlights trending data that may not be immediately obvious when observing a periodic report, or may not be entirely clear from periodic alerts. For example, the trend in the results shown in FIG. 8 may indicate that there was a learning curve to using the DES, and that DES implantation is becoming safer. Such trending information may be used to dynamically change the expectation. For example, if some portion of the deaths resulting in the early part of the interval can be attributed to unfamiliarity with the device, procedure, or patient selection, a more accurate indication of the safety of the device itself may be reflected by lowering the expectation as the implantation procedure becomes more mature.

Accordingly, periodic and cumulative analyses may be used together to, amongst other things, identify both recent trends and overall trends in event rates. Evaluation of recent trends may have reduced power to detect significant shifts in event rates, due to the reduced sample size. However, periodic monitoring may serve as a very useful early warning indicator when, for example, cumulative event rates have yet to cross the alert threshold. Early warnings may be beneficial to encourage increased monitoring of the medical entity and to heighten awareness as to a potential problem.

Referring again to FIG. 4, method 12 includes a risk stratified SPC expectation analysis. As discussed above, risk stratification includes separating the data according to a predetermined criteria, so that the data may be analyzed according to the classification. The various data sources, whether voluntary or mandatory, may be populated by participating institutions filling out forms or records for each instance of the medical entity. For example, for each patient receiving a medical device, procedure or drug, a record in the corresponding database may be filled out. A record may include the patient's name or (for anonymity) a number, the patient's gender, age, and the type of medical entity applied (e.g., the type of DES used). The record may also include information relating to other diseases or complications, such as diabetes, asthma, etc. In the case of a drug, the record may include other drugs that the patient is currently taking. It should be appreciated that the content of the record may depend on the type of information being reported and may be of any type and of any content, as the aspects of the invention are not limited in this respect.

In one embodiment, the information may be used to formulate a plurality of risk strata. For example, the patients may be categorized as low, medium or high risk. A set of rules may then be defined that determines which risk category or strata a patient belongs to. For example, age may be a indicator of risk. Accordingly, the risk strata may be defined purely on age. For instance, rules such as, if the age is less than 60, the patient may be categorized as low risk. If the age is greater than or equal to 60 and less than 80, the patient may be categorized as medium risk, and if the age is greater than or equal to 80, the patient may be categorized as high risk. Other information may be used to establish rules of any complexity. For example, whether a patient has diabetes may effect the risk level of the medical entity and may be incorporated into the rules. An exemplary rule may be that if the patient's age is less than 50, but has diabetes, the patient may be categorized as medium risk, instead of low risk as in the example above. Any number of factors may be used to establish rules to categorize patients into any number of strata, as the aspects of the invention are not limited in this respect.

It should be appreciated that the rules and the type of information used to define the risk strata may depend on the type of medical entity, and on what factors may be valuable in determining risk strata and in defining useful categories for the analysis. In addition, it should be appreciated that the strata need not be defined to categorize risk. For example, a practitioner may be interested in determining the effects of a particular medical entity based on gender, ethnicity, etc., independent of the associated risk. This information may assist in identifying a problem that pure risk stratification may miss, or not so clearly indicate. Accordingly, the data may be separated or categorized in any manner to achieve stratification, as the aspects of the invention are not limited in this respect.

SPC expectation analysis may then be performed on the data, in view of the risk strata, either periodically or cumulatively by any of the methods described above. In particular, different expectations may be set with respect to each strata. Moreover, different confidence intervals may be assigned to the expectations of the respective strata. For example, the low risk strata may be assigned a lower expectation of event rates (e.g., death) than the medium and high risk categories. However, empirical evidence may suggest that the variability of the low risk category is also less, such that a narrower confidence interval (e.g., 97%) may be tolerated for the low risk category. The expectation and thresholds may be set in any manner for the various defined strata, as the aspects of the invention are not limited in this respect.

Referring back to FIG. 4, method 13 includes a logistic regression (LR) analysis that incorporates risk adjustment. Logistic regression can be used to predict a dependent variable (e.g., the probability of an event occurring) on the basis of continuous and/or categorical independent variables (e.g., factors such age, gender, other health issues, etc.) and to determine the percent of variance in the dependent variable (e.g., a confidence interval) explained by the independent variables, to rank the relative importance of the independent variables, to assess interaction effects, and to understand the impact of covariate control variables. Accordingly, LR techniques can provide a probability of outcome, based on multiple variables, on a case-level basis.

Logistic regression typically applies maximum likelihood estimation, which estimates the probability of a certain event occurring. Accordingly, the probability of an event occurring can be used as an expectation of the rate of event occurrence. Similarly, the variance may be used to establish a confidence interval. LR analysis is based on a predictive model taking into consideration the significance of one or more covariate factors. The form of the logistic model can be expressed as:

$$P = \frac{1}{1 + {ⅇ}^{- ( {B}_{0} + {B}_{1} {X}_{1} + {B}_{2} {X}_{2} + ⋯ {B}_{k} {X}_{k} )}} \qquad ( 1 )$$

Where B₀ is a constant (i.e., the intercept) and Bᵢ are coefficients of the predictor or covariate variables Xᵢ (i.e., the factor). The computed value, P, is a probability in the range from 0 to 1. Accordingly, the probability is a function of the contribution of weighted factors forming a predictive model. The weighting (i.e., the values of the Beta coefficients) may be derived from any available information such as empirical data, published test results, expert opinion, etc. It should be appreciated that a single or multiple Beta values may be used to form the predictive model, and the number used may depend on the type of analysis being performed. Other predictive model formulations may be used as well, as the aspects of the invention are not limited in this respect.

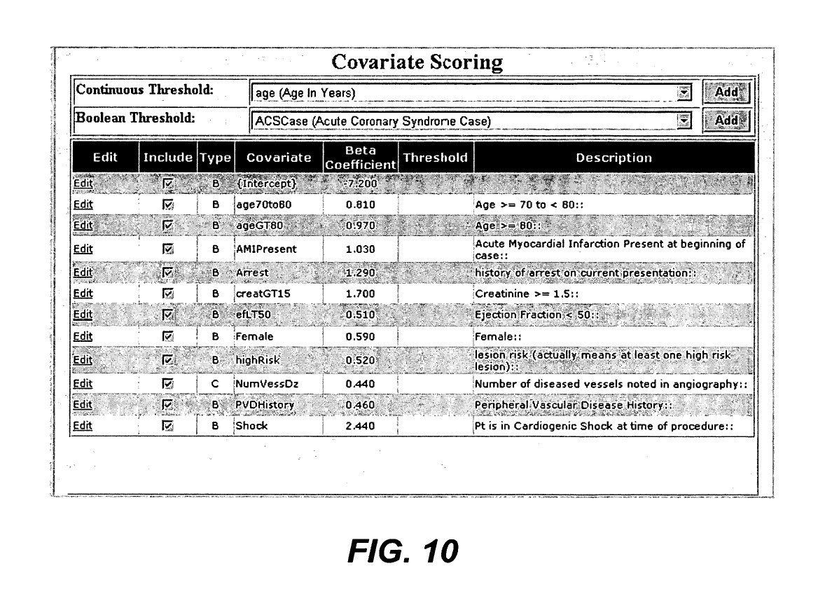

FIG. 10 illustrates a covariate scoring for an LR model for determining the probability of SAT given the listed factors, in accordance with some embodiments of the present invention. In particular, in the “Description” column, various factors that are considered relevant to developing acute coronary syndrome as a result of DES implantation are listed. In the “Beta Coefficient Column,” the weights for each factor may be input by an operator or administrator of the analysis. As discussed above, LR may be based on predictive models derived from previous empirical data, expert opinion, etc. Accordingly, the values of the beta coefficients are based on the predictive model for the particular analysis being performed.

It should be appreciated that different analyses corresponding to different medical entities will have a wide range of factors that may be important. The list of factors and the selected beta factors illustrated in FIG. 10 are merely exemplary of one analysis for a particular medical entity. Any number and type of factors may be used in deriving a predictive model for a given analysis of a medical entity, as the aspects of the invention are not limited in this respect.

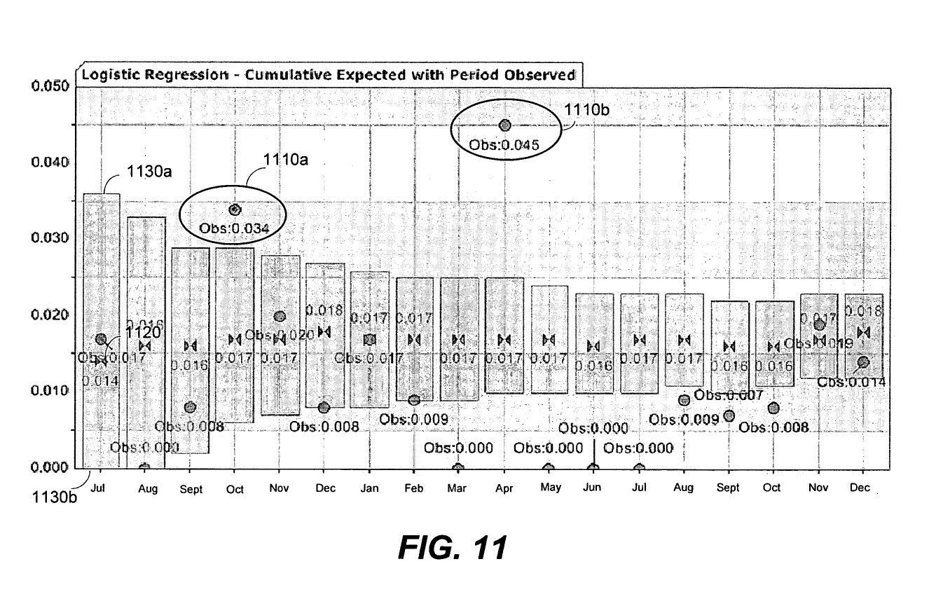

FIG. 11 illustrates results of an LR analysis of cases of SAT resulting from the use of a DES device, in accordance with some embodiments of the present invention. In FIG. 11, the expectations are denoted by the pair of triangles, for example, expectation 1120 for the month of July. The confidence intervals are denoted by the bars, for example, the bar formed by upper boundary 1130a and 1130b for the month of July. The actual observed event rates are denoted by the circles, for example, the observed event 1110a for October. In the analysis performed in FIG. 11, the observed rates for October and April exceeded the upper bound of the confidence interval and therefore generated an alert to indicate the occurrence of an adverse event. In the analysis performed in FIG. 11, the expectation and confidence intervals are computed cumulatively, and the observed event rates are computed periodically (i.e., over month epochs). It should be appreciated that the observed event rates may be computed cumulatively as well, as the aspects of the event rates are not limited in this respect.

It should be appreciated that since the probability of an event occurring is computed as new data is received, the corresponding expectation is adjusted according to the new data. Accordingly, risk adjustment occurs implicitly using LR analysis. Similarly, since the amount of data used in LR computations increases as new data is received, the variance in the data is also subject to change. For example, the LR results illustrated in FIG. 11 show a steady narrowing of the confidence interval and some (though not marked) fluctuation in the expectation. Typically, the more data available, the higher the signal-to-noise ratio (SNR), and the less vulnerable the outcomes are to outliers. As a result, the variance and the corresponding confidence intervals may become smaller as more data is received, as was the case of the analysis performed and illustrated in FIG. 11. Therefore, the evolving analyses of LR may provide accurate assessment of actual adverse events due, in part, to the fact that the LR analysis adapts to new data.

Referring back to FIG. 4, method 21 includes a Bayesian Update System (BUS) to perform uniform inference statistical analysis. BUS methods incorporate Bayes' theorem into a SPC framework by utilizing prior observed data to evolve the estimates of risk. Alerting boundaries may be calculated by several methods. In a first method, previous current study data with the prior data of the SPC method are used to calculate a desired confidence interval (e.g., a 95% confidence interval). As a result, alerting boundaries shift during the course of real-time monitoring due to the influence of the earlier study data.

In a second method, alerts are based on the evolution of updated risk estimates represented as probability density functions (PDF). In each period or epoch, a new PDF is generated based on the cumulative study event rate and baseline event rate. Alerting thresholds are generated by specifying minimum overlap of the distributions (e.g., by comparison of central posterior intervals). The first comparison PDF is the initial prior PDF, and the second PDF is the previous period's or epoch's PDF.

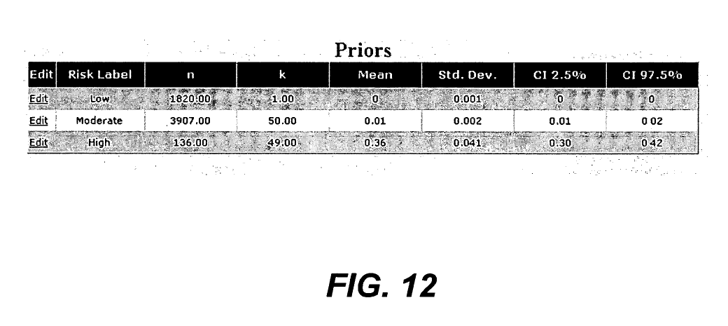

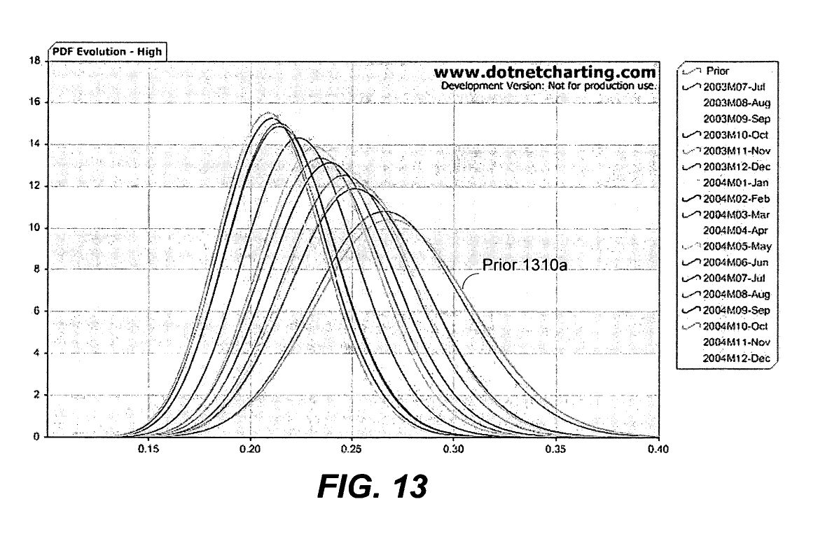

Priors for purposes of the Bayesian analysis may be obtained from any available empirical data, SPC results, previous publications and/or test results, etc. FIG. 12 illustrates priors established for an initial prior PDF using the second method described above (i.e., defining alert thresholds by minimum overlap in prior and posterior PDF's). As shown, the Bayesian analysis to be performed is stratified. That is, priors are defined for a low, medium and high risk category. It should be appreciated that the Bayesian analysis may be performed uniformly without stratification by a single representative prior. FIG. 13 illustrates the evolution of the PDF's for the high risk strata defined in FIG. 12. PDF 1310a is the initial prior distribution. The cumulative PDF's for each epoch (i.e., for each month) are computed and illustrated as they evolve over the course of 18 months, according to one study of a DES.

BUS methods may be particularly useful in reducing the impact of early outliers in data, and may facilitate more accurate alerting when a limited amount of data exists (e.g., during intervals near the time the medical entity is released to the public.) BUS methods may provide a useful complement to other statistical methods as described in further detail below. In particular, alert algorithms based on BUS methods may be combined with one or more other alert methods (e.g., SPC and/or LR methods) to take advantage of the strengths and weaknesses of the each method.

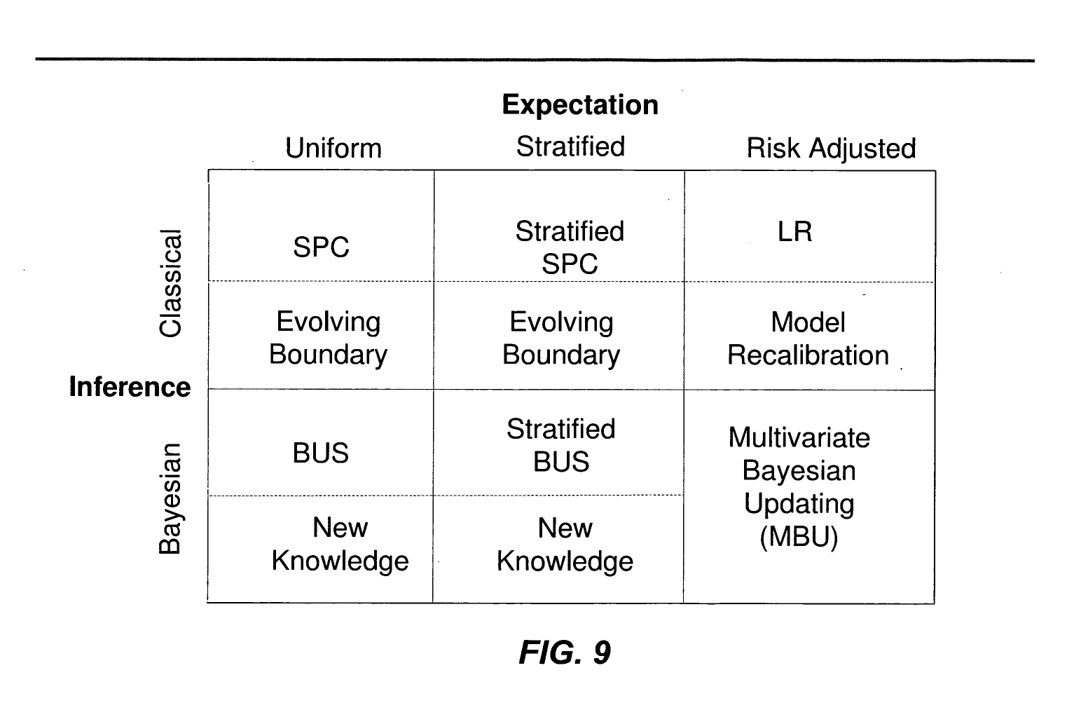

FIG. 9 illustrates various statistical methods that may be used in a safety monitoring system, in accordance with some embodiments of the present invention. The methods illustrated in FIG. 9 may be similar to the methods illustrated in FIG. 4. However, the methods may be supplemented using information obtained during the analysis (e.g., as the data becomes available and accumulates) to adjust the parameters of the analysis. For example, for SPC, information gleaned during the analysis may be used to adjust the expectation of the analysis, either in the uniform case or for each category in a stratified analysis. Similarly, information from an LR analysis may be used to adjust the LR model (e.g., to adjust weights of the beta coefficients). Likewise, new knowledge obtained from a BUS analysis may be incorporated into the Bayesian framework. It should be appreciated that the additional information used to adjust one or more analyses need not be obtained from a current analysis, but may be obtained from outside sources, such as recent publication of empirical data, test results, etc., as the aspects of the invention are not limited in this respect.

The various alerting methods may have particular strengths and may also be vulnerable in some instances. Applicant has appreciated that by combining the various alerts, a meta-alert may be developed that provides a more accurate indication of when a true adverse event has occurred. Two characteristics of an alert that may be important include high sensitivity and high specificity. High sensitivity may be important for high risk events such as mortality, catastrophic device failure, etc. In particular, for high risk events, false positives may be tolerated to avoid missing a true adverse event. High specificity may be important for lower risk, low morbidity events such inflammatory or allergic reaction, non-fatal side-effects, etc. In these instances, it may be more important to only raise an alert when a true adverse event transpires. Other characteristics of alerts may be valuable and may be exploited by combining alerts generated by multiple analysis strategies.

In some embodiments according to the present invention, a combination of Bayesian Updating and SPC alerts are combined to optimize sensitivity and specificity for general safety surveillance. One exemplary meta-alert includes an alert rule that instructs the meta-alert to be generated if a Bayesian alert fires in one or more periods in an observation interval and an SPC alert fires in more than one third of the periods in the observation interval. It should be appreciated that any one or combination of Bayesian, SPC and LR alerts may used to define a meta-alert. The monitoring module may expose one or more interfaces that allow a user or operator to customize a meta-alert for a particular analysis using one or any combination of available alerts from available statistical processes. That is, an operator may be enabled to combine, weight and/or set up a series of rule alerts that define a meta-alert that may facilitate accurate detection of adverse events. The concept of combining, weighting or otherwise customizing alerts based on a set of rules may be applied to any type of alert or in any combination, as the aspects of the invention are not limited in this respect.

Various aspects of the invention derive from applicants appreciation that important information may be gleaned by analyzing safety data from a variety of sources. Obtaining information from the various data sources, in combination with a centralized data warehouse (e.g., the data warehouse described in connection with FIGS. 2 and 3), may be achieved by implementing a safety monitoring system as a distributed network application. In this manner, the actual data sources and the data warehouse may be physically located anywhere. Similarly, updates to the data sources and/or data warehouse would not need to be propagated to local copies of the database.

To facilitate the distributive safety monitoring system, the safety monitoring module may be a network application. The front-end of the safety monitoring module may be a web-based application using an interface such as a browser. Accordingly, operators could access the database information, construct analyses, customize tests, view results, receive alerts, etc. through the browser installed on the operator's computer, while most of the distributive application resides elsewhere.

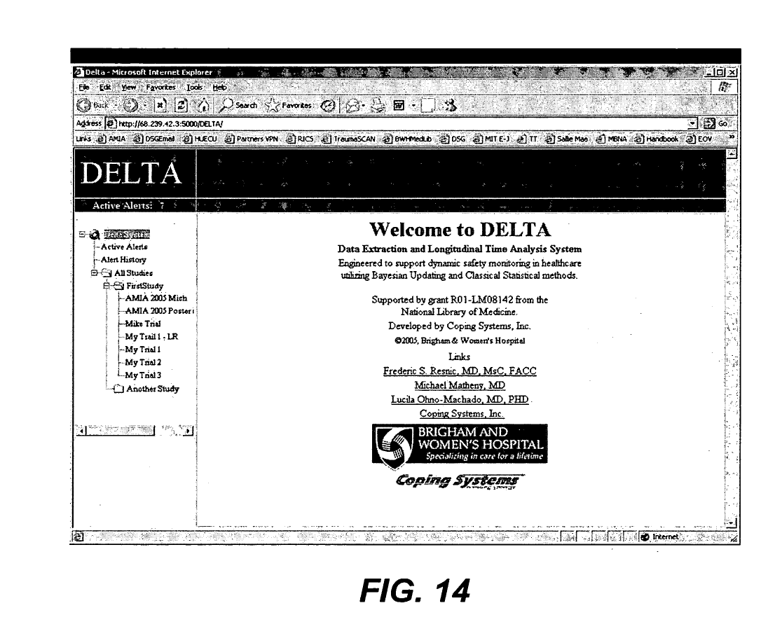

FIG. 14 illustrates a screen shot of an introductory page of a browser based interface provided to the operator to interact with the safety monitoring system, in accordance with some embodiments of the present invention. The safety monitoring system of this embodiment is the Data Extraction and Longitudinal Time Analysis (DELTA) system developed by the Applicant. As illustrated in FIG. 14, the DELTA system is operated via the familiar Microsoft® Internet Explorer web browser and was developed using the Microsoft® .NET environment. Along the left side of the screen, a file folder hierarchy is provided. The file folder hierarchy may be arranged and customized to suit the operator.

The root folder includes all the available information available through the DELTA system. If the hierarchy is expanded, folders representing the available data sources are listed as the next level. For example, the folder labeled “All Studies” may include all the safety information available to the system. Underneath, each folder in the hierarchy may represent a data source, or all of the information on a particular medical entity from a variety of data sources. Under each study (e.g., data available on a particular medical entity), the folders at the next level correspond to individual analyses that are being performed on the associated data. Accordingly, the DELTA system supports performing multiple analyses on multiple medical entities simultaneously.

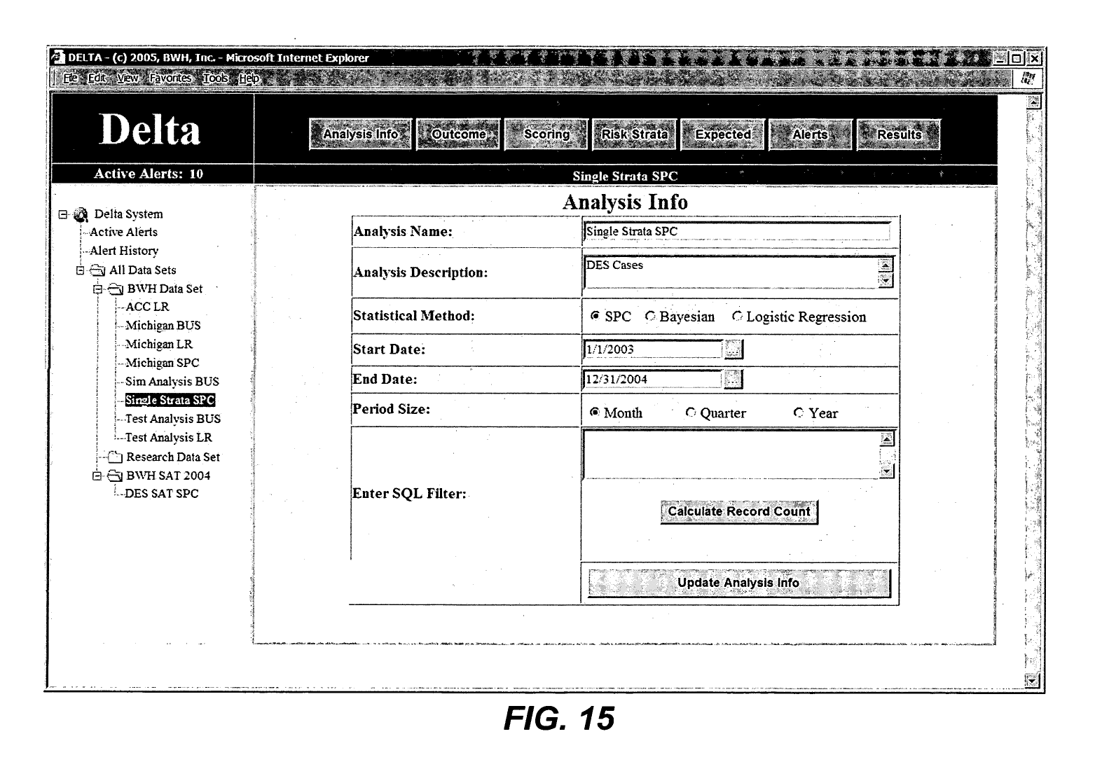

FIG. 15 illustrates the interface provided to establish and customize one or more analyses of the data, in accordance with some embodiments of the present invention. In this embodiment, the DELTA system is used. In FIG. 15, two data sets are available. Under the first data set labeled “BWH data set” a number of analyses are being performed. The operator has opened and selected the single strata SPC analysis. As shown by the selected radio button, this analysis is an SPC analysis, and more particularly, a uniform single strata SPC periodic analysis, similar to that discussed above in FIG. 4 with respect to method 11. As shown in the “Analysis Description,” this data includes reporting information about drug eluting stents.

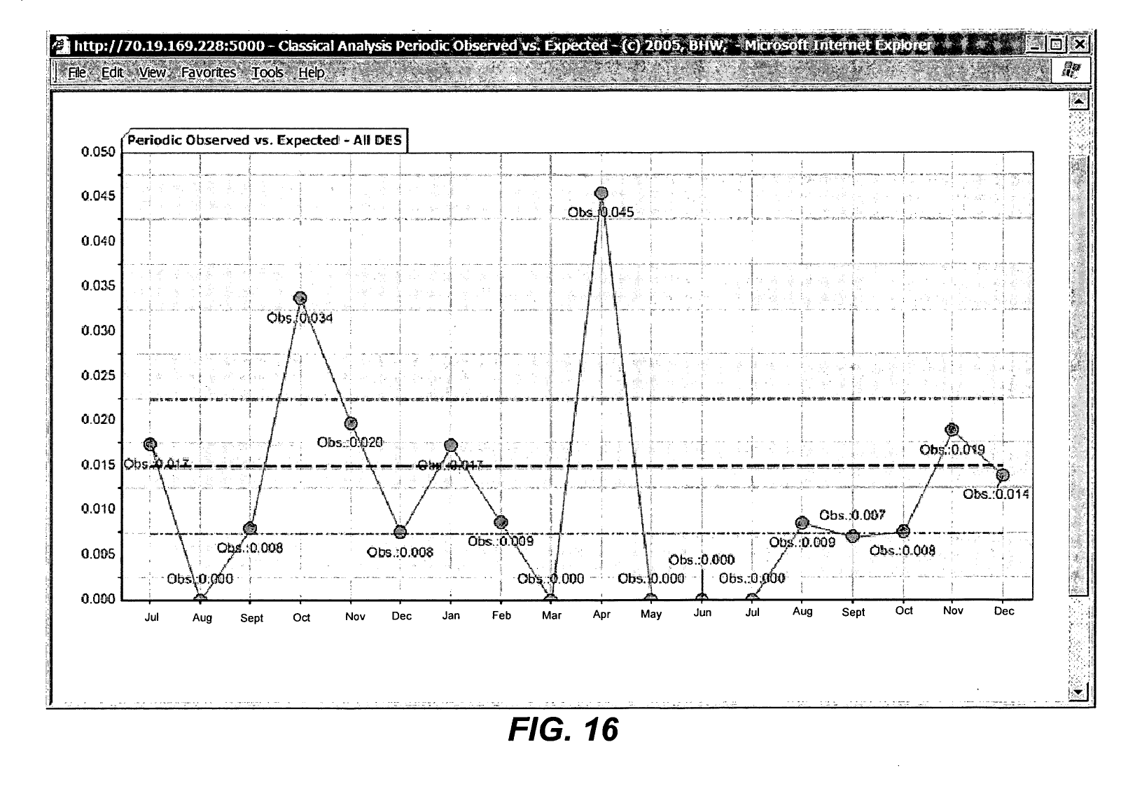

Along the top of the screen shot in FIG. 15 are a number of available buttons that allow the operator to customize the analysis. In order to set up the analysis that resulted in the graph illustrated in FIG. 6, for example, the “Expectation” button may be pressed and the desired expectation entered. To stratify the analysis, the “Risk Strata” button may be selected and the strata defined and the rules of categorization input into the analysis. FIG. 16 illustrates a screen shot of a report of this analysis. It should be appreciated that the October and April results generated an automatic and real-time alert, for example, by creating a pop-up window or by sending an email to the operator to notify him or her that an alert was generated, indicating a possible adverse event.

Various other features of the DELTA system will be described below in connection with analyses of the risk of retroperitoneal hemorrhage with the use of a vascular closure device. In particular, reporting information for a particular vascular closure device may be collected at one or more data sources (e.g., one or more data sources 310 illustrated in FIG. 3). The data warehouse (e.g., data warehouse 320) may consolidate the information from the variety of sources and format the data so that it can be queried by the monitoring module (e.g., monitoring module 350), which, in some embodiments, is the DELTA monitoring module. The various analyses established through the DELTA interface may operate in real-time to provide a continuous analysis of the data. For example, when one or more of the data sources is updated, the data warehouse performs a corresponding update and the analyses established by the DELTA monitoring module are performed in view of the new data.

It should be appreciated that the operator can select when and under what circumstances the analysis is performed. For example, the operator may choose various analyses to be performed upon any change in the information in any of the corresponding data sources. Alternatively, the operator can choose to have the analyses performed on a periodic schedule, such as a daily, weekly, monthly analyses, etc. Accordingly, continuous (as opposed to retrospective) analyses may be performed on the reporting information available on one or more medical entities.

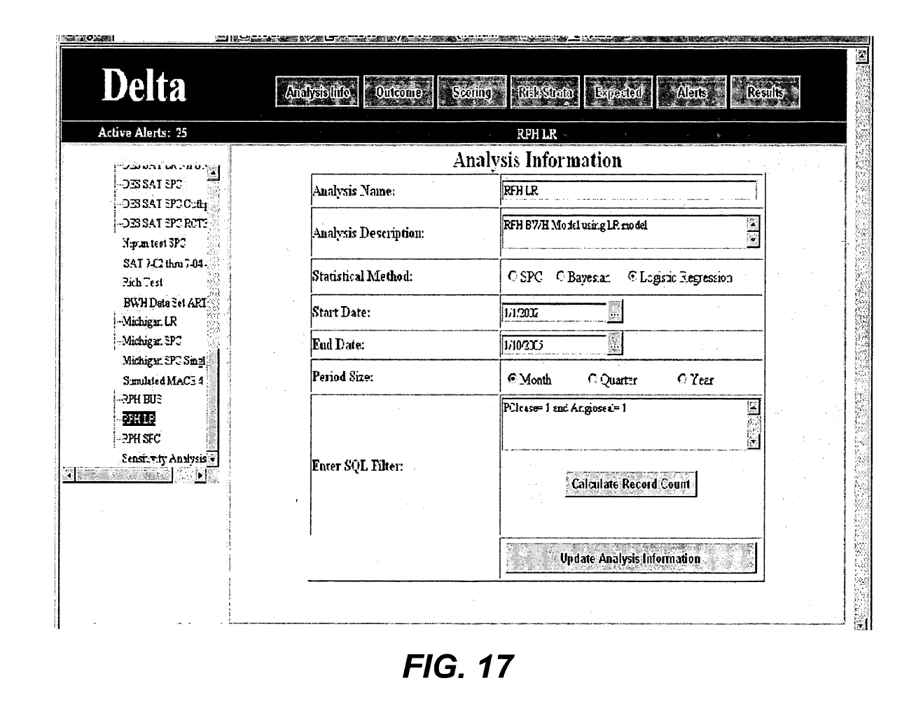

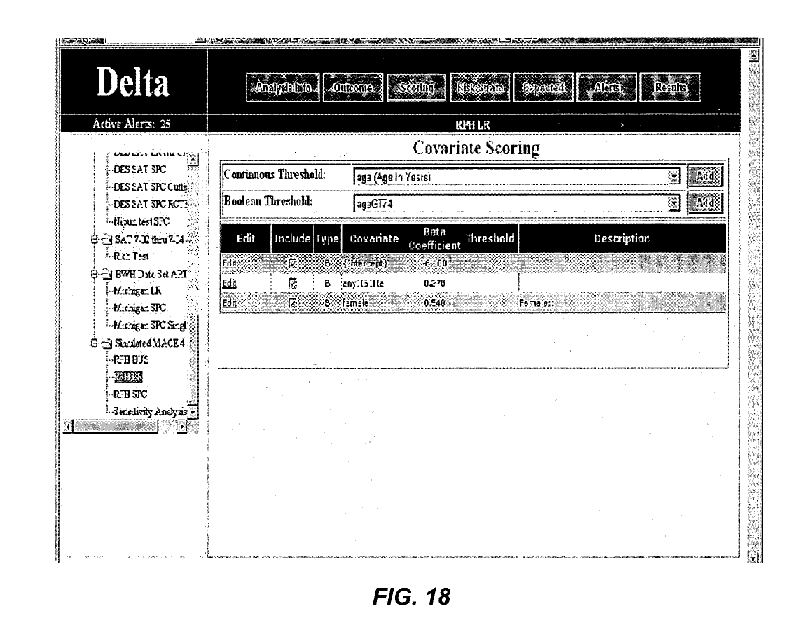

FIG. 17 illustrates the instantiation of an LR analysis on the vascular closure device, in accordance with some embodiments of the present invention. Accordingly, the screen shot is of the “Analysis Information” page. It should be noted that, since the selected analysis is an LR analysis which implicitly includes risk information and expectation, the “Risk Strata” and “Expectation” buttons are not activated since this information may not need to be provided by the operator. FIG. 18 illustrates the screen shot for establishing the weightings for the desired beta factors, as described above in connection with Equation 1, in accordance with some embodiments of the present invention. Here, two beta factors were considered relevant. The intercept (i.e., B0) is set at −6.1. The beta coefficient for the use of the anticoagulant class glycoprotein 2b3a inhibitor, is set at 0.270 and whether the patient is female or not is set at 0.540. As discussed above, the covariate scoring may be based on previous data, published test results, expert opinion, etc.

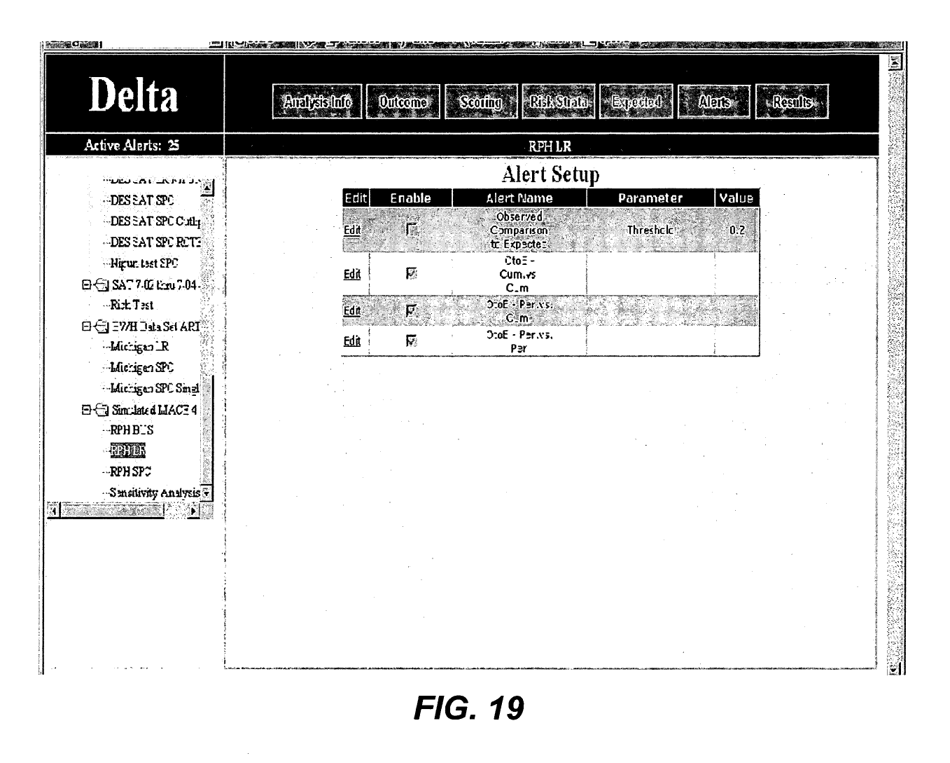

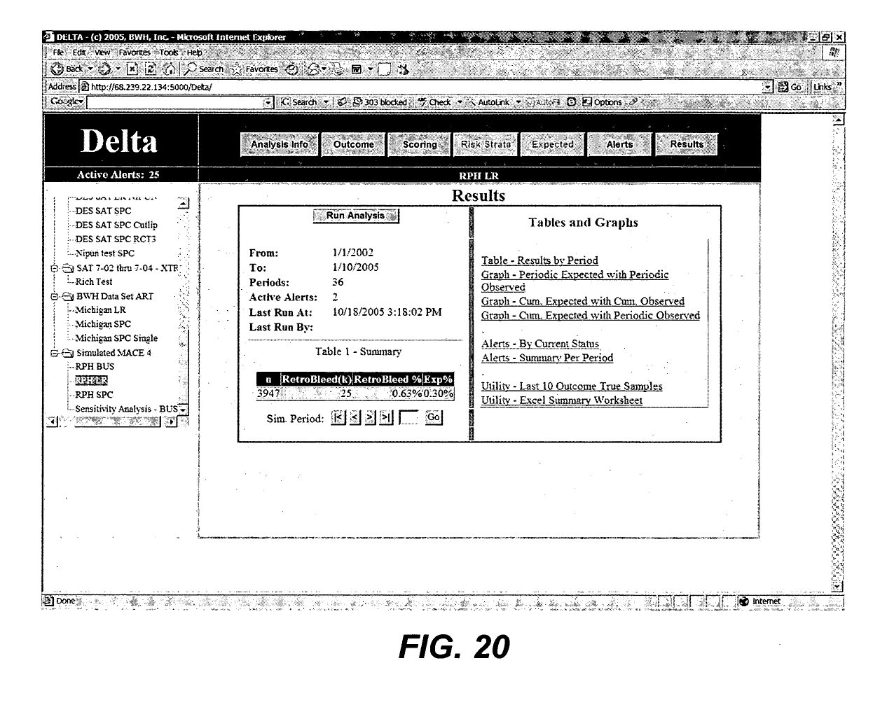

FIG. 19 illustrates the alert structure set up for the LR analysis, in accordance with some embodiments of the present invention. FIG. 20 illustrates the page generated when the “Result” button is selected. The page provides a summary of the analysis performed up until that date. For example, the page provides the number of periods or epochs over which the analysis has been performed, the number of alerts that have been generated, a summary of the analysis parameters, the data of the last time the analysis was performed, etc. The result page also provides the capability for the operator to manually request the analysis be performed. For example, the analysis may be set to automatically perform an analysis on a monthly basis. However, the operator may want to run the analysis at some point during the month, and may select the “Run Analysis” button to perform the analysis on the new data that may have been entered since the last run of the analysis.

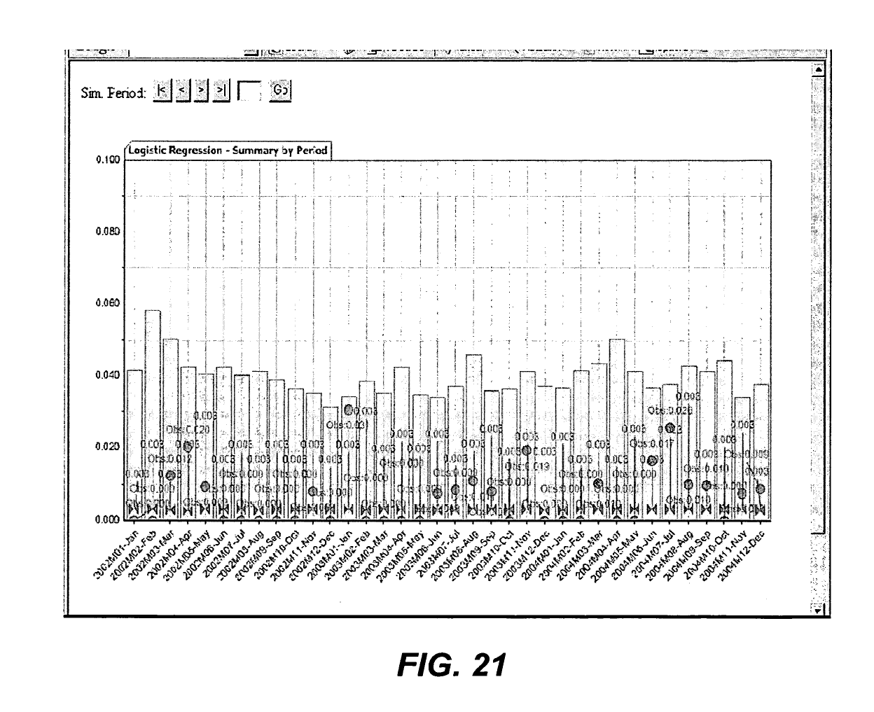

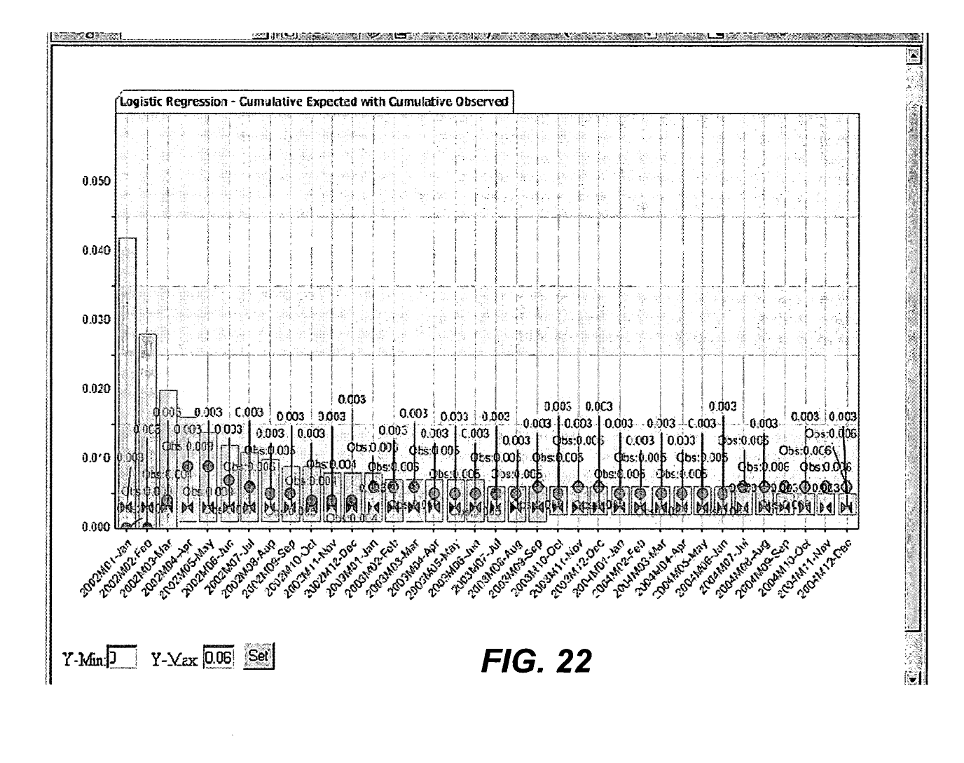

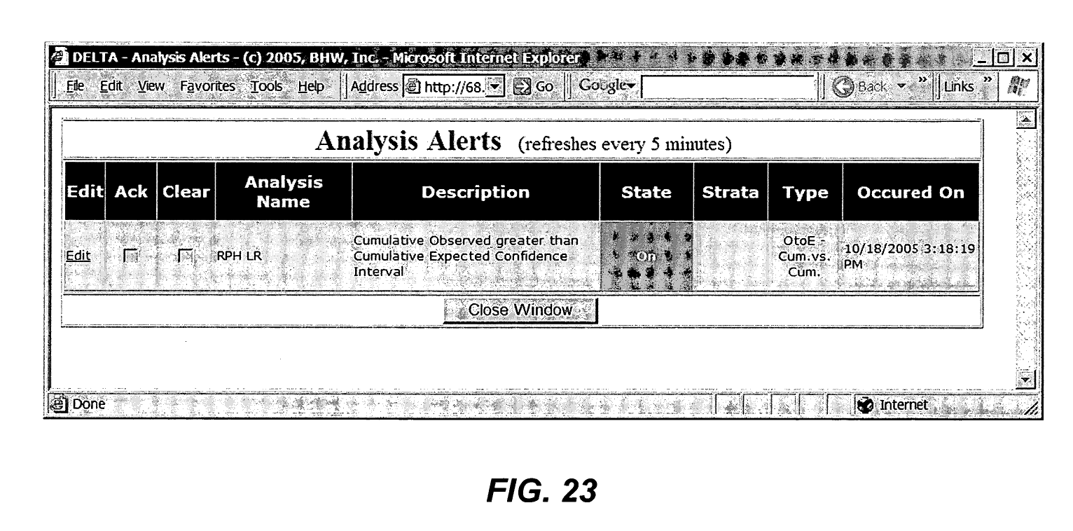

In the right hand column, a list of current reports are shown, which are provided as live links for viewing the reports. For example, FIG. 21 illustrates the “Summary per Period” report. Similarly, FIG. 22 illustrates the “Cumulative Expected with Cumulative Observed” report. Any number of reports of any type may be generated to facilitate visualization of analysis results. FIG. 23 illustrates the alerts that have been defined for the LR analysis, in accordance with some embodiments of the present invention. Here, a single alert has been defined, namely, an alert is generated each time the observed rate of retroperitoneal hemorrhages resulting from the vascular closure devices exceeds the expectation interval resulting from the LR model.

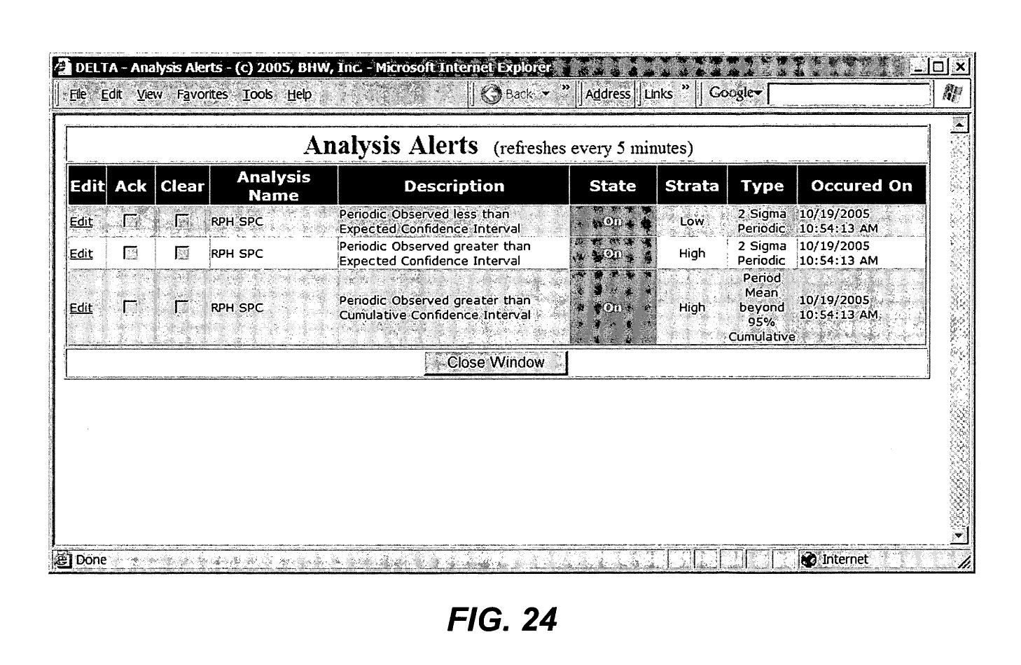

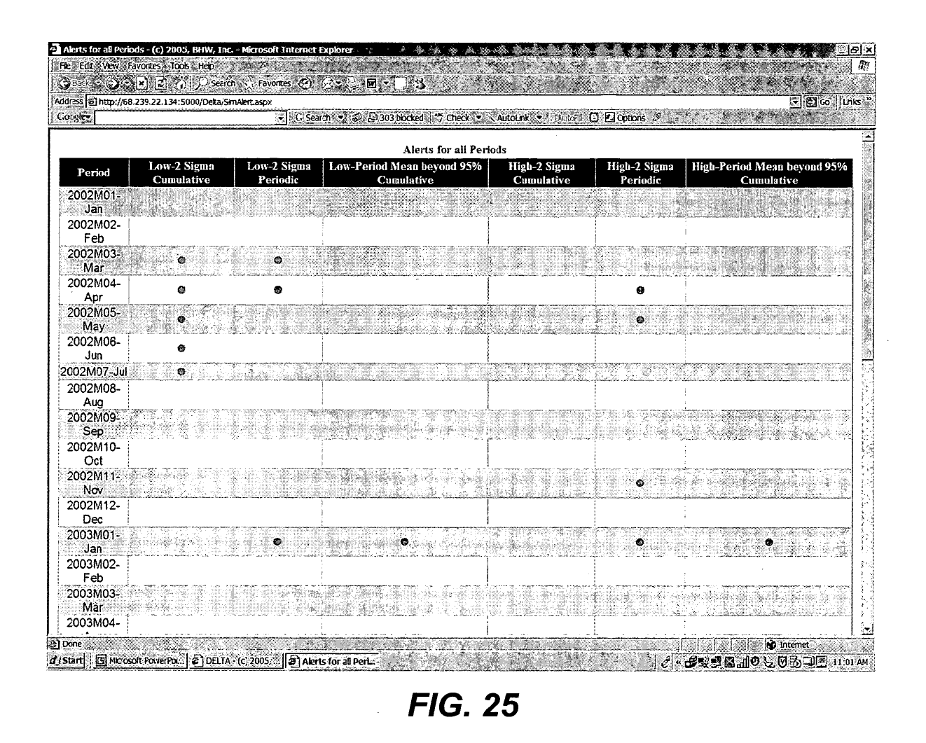

FIGS. 24-27 illustrate components of a stratified SPC analysis of the vascular closure device. In particular, the stratified SPC analysis was defined with a low risk and high risk category. FIG. 24 illustrates the alert structure established for the SPC analysis. In particular, three alerts have been set for the SPC analysis. The first alert corresponds to the low risk strata. The alert rules are set such that each time the periodic observed number of retroperitoneal hemorrhages is less than the lower bound of the confidence interval an alert is generated. The second and third alerts correspond to the high risk strata. The alert rules for the second alert assert that each time the periodic observed number of retroperitoneal hemorrhages is greater than the upper bound of the confidence interval of the established expectation, an alert is generated. The alert rules for the third alert assert that each time the periodic observed number of retroperitoneal hemorrhages is greater than the cumulative confidence interval, an alert is generated. FIG. 25 illustrates the alerts that were generated from the above alert structure for all the periods over an 18 months interval, in tabular form.

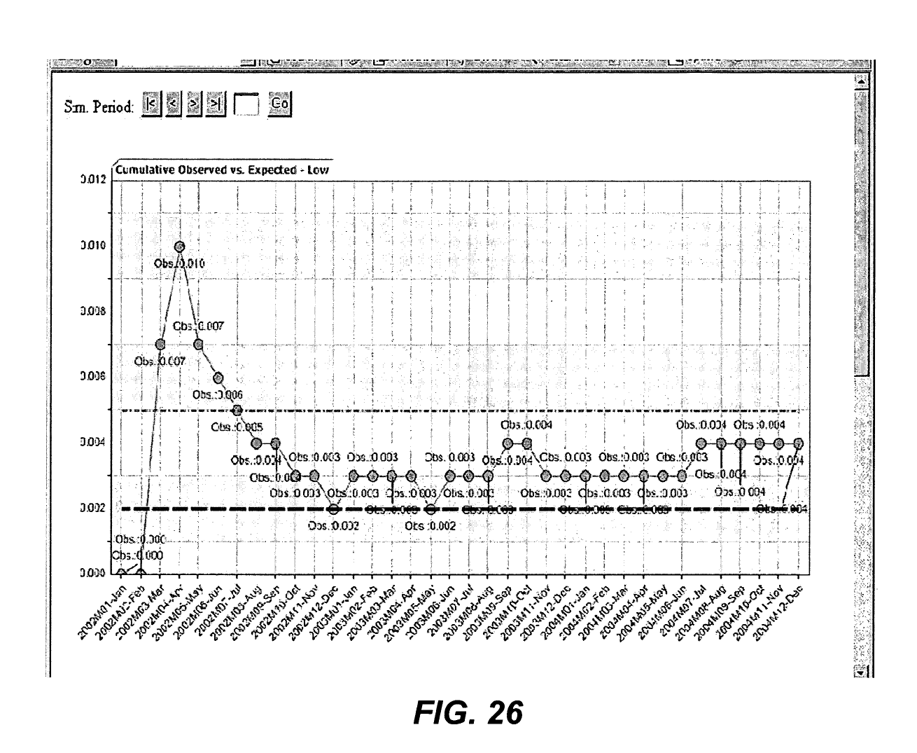

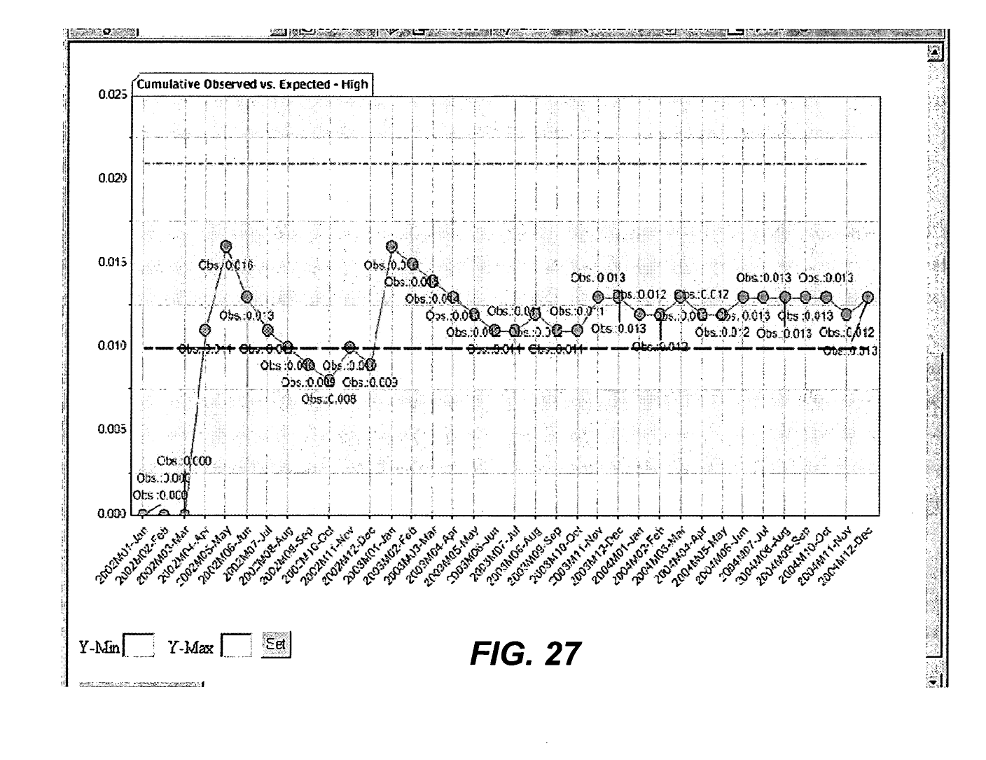

FIGS. 26 and 27 illustrate reports generated from the DELTA SPC analysis. FIG. 26 illustrates results showing the cumulative observed data versus the expectation and confidence intervals for the low risk strata. Similarly, FIG. 27 illustrates results showing the cumulative observed data versus the expectation and confidence intervals for the high risk strata. FIGS. 26 and 27 illustrate exemplary reports that may be generated using the DELTA software. However, it should be appreciated that any type of report or data visualization may be generated, as the aspects of the invention are not limited in this respect.

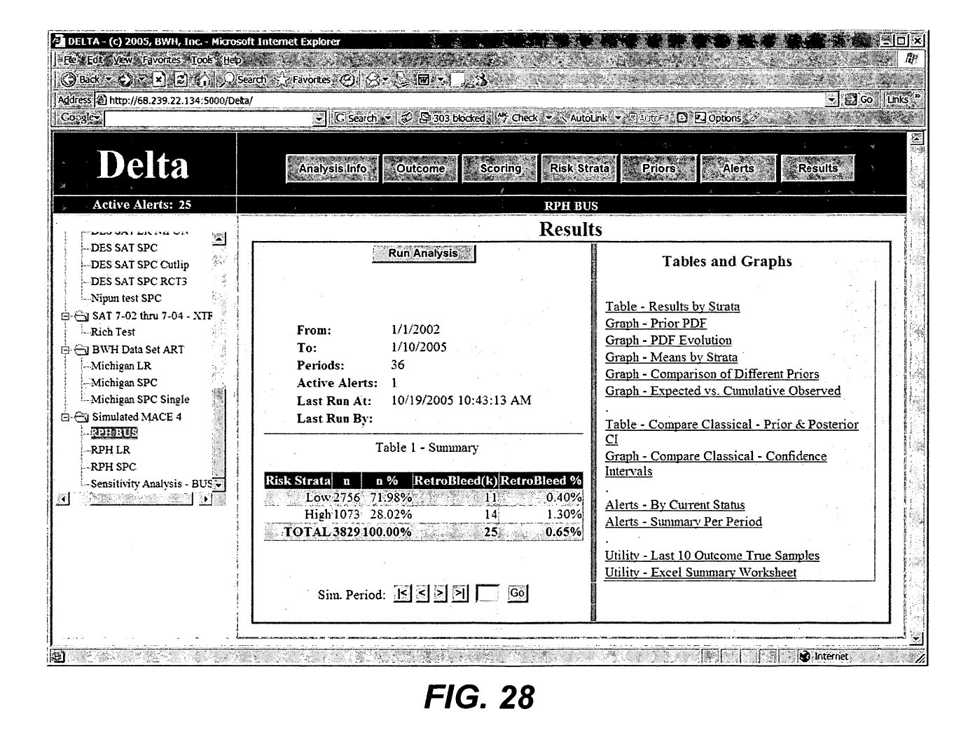

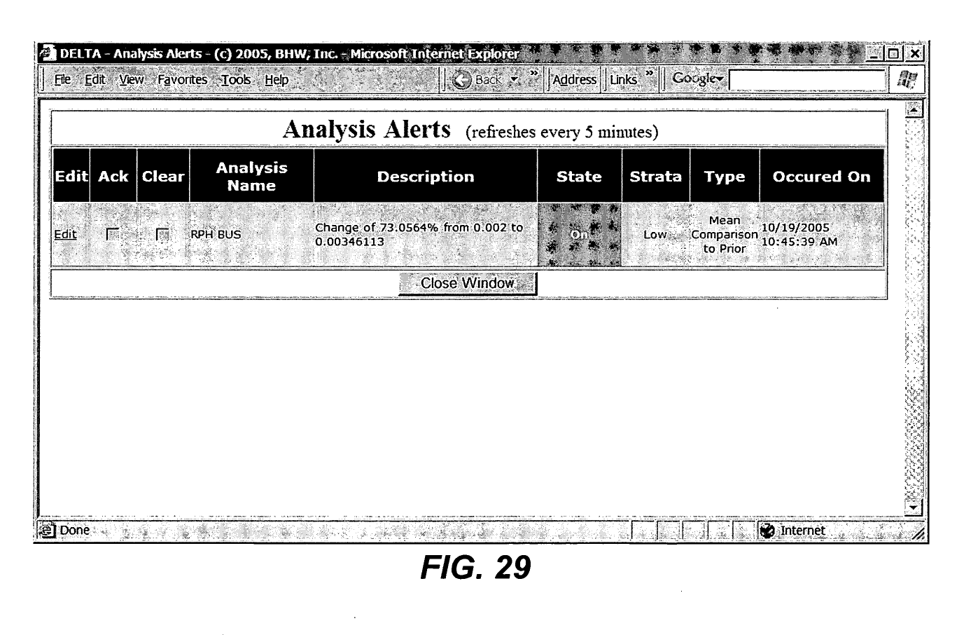

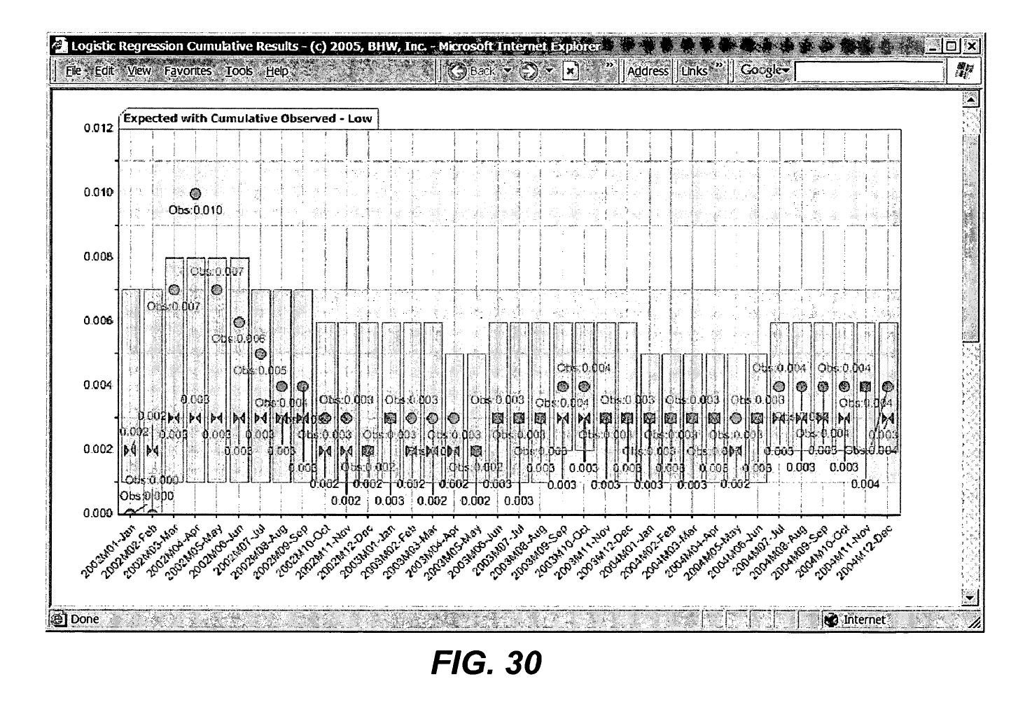

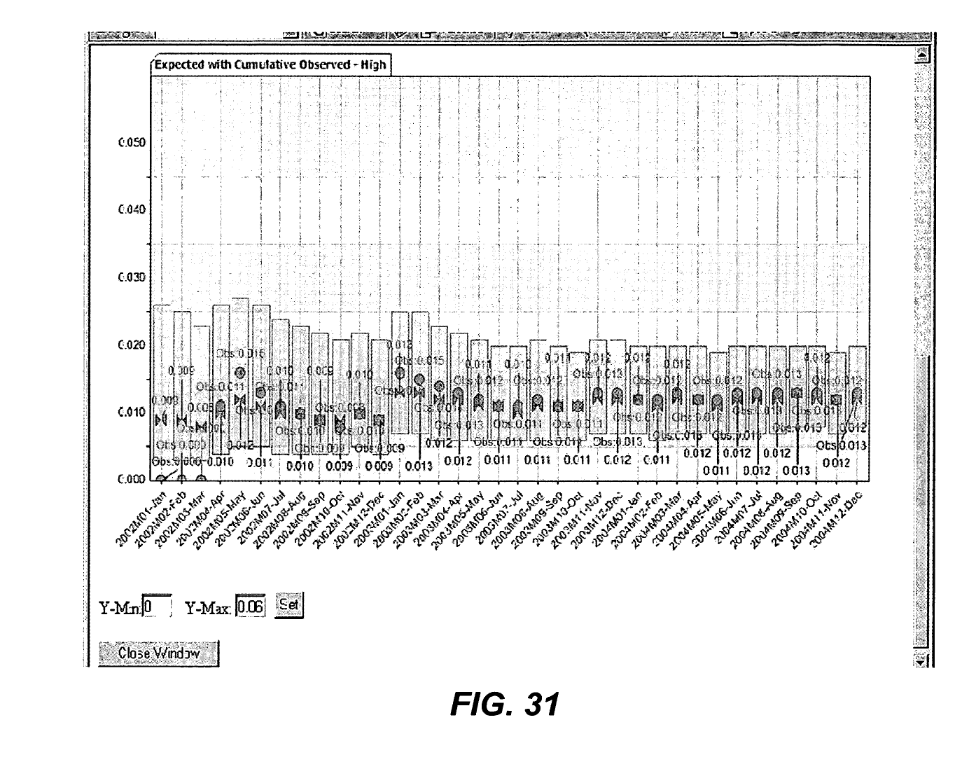

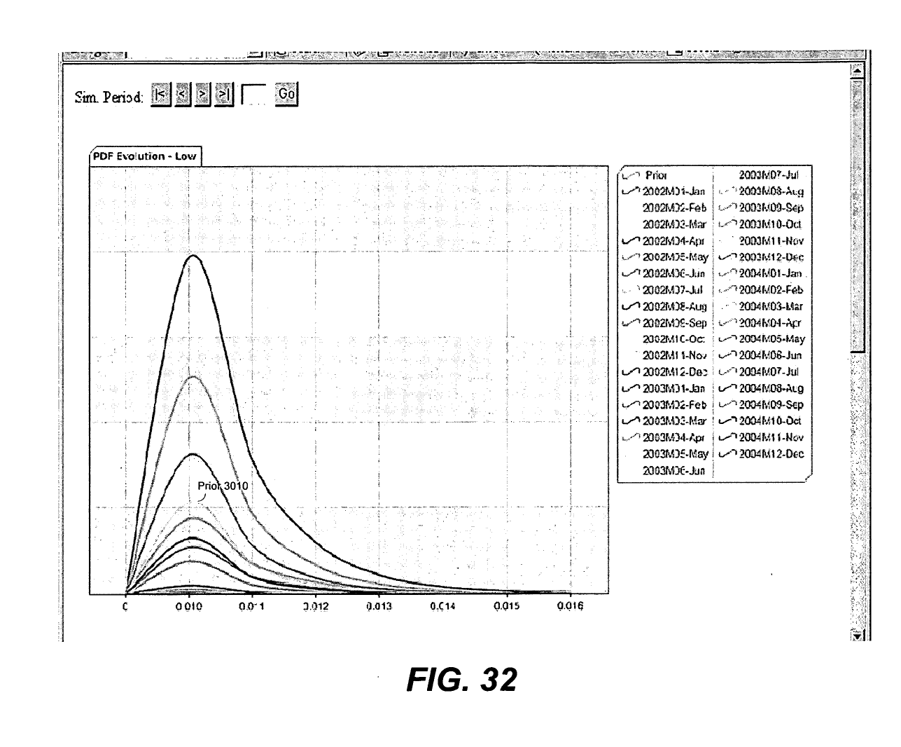

FIGS. 28-32 illustrate components of a stratified Bayesian Updating System (BUS) analysis performed on the vascular closure device information, in accordance with some embodiments of the present invention. FIG. 28 illustrated the results page for an established BUS analysis, having a low risk and high risk strata. As shown in the summary, one alert has been defined for this analysis, which is illustrated in FIG. 29. In particular, the alert defined in FIG. 29 is based on a mean comparison with the prior PDF. Whenever a change in the observed posterior distribution exceeds the minimum threshold, an alert is generated. FIG. 30 illustrates the report of cumulative observed data versus the expected for the low risk strata. FIG. 31 illustrates the report of cumulative observed data versus the expected for the high risk strata. FIG. 32 illustrates the cumulative PDF's evolution for the low risk strata over a 3 year interval.

As illustrated above, numerous analyses using various statistical methods and alert structures were used on the same information. In some embodiments according to the present invention, the safety monitoring module is adapted to track an arbitrary number of analysis and prompt alerts to an arbitrary number of potential adverse events. It should be appreciated that any source of data relating to any medical entity may be used, and any type of statistical analysis may be performed to continuously analyze the information, as the aspects of the invention are not limited in this respect.

As should be appreciated from the foregoing, there are numerous aspects of the present invention described herein that can be used independently of one another or in any combination. In particular, any statistical method may be employed in any of numerous combinations and procedures. Moreover, any type of alert structure or formulation of one or more meta-alerts may be used with any type of analysis. It should also be appreciated that in some embodiments, all of the above-described operations can be used together in any sequence, or any combination or subset of the operations described above can be employed together in a particular implementation, as the aspects of the present invention are not limited in this respect. In addition, the various operations may be performed on any type of device or apparatus, and are not limited to any particular implementation.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

It should be appreciated that the various methods outlined herein may be coded as software that is executable on one or more processors that employ any one of a variety of operating systems or platforms. Additionally, such software may be written using any of a number of suitable programming languages and/or conventional programming or scripting tools, and also may be compiled as executable machine language code. In this respect, it should be appreciated that one embodiment of the invention is directed to a computer-readable medium or multiple computer-readable media (e.g., a computer memory, one or more floppy disks, compact disks, optical disks, magnetic tapes, etc.) encoded with one or more programs that, when executed, on one or more computers or other processors, perform methods that implement the various embodiments of the invention discussed above. The computer-readable medium or media can be transportable, such that the program or programs stored thereon can be loaded onto one or more different computers or other processors to implement various aspects of the present invention as discussed above.

It should be understood that the term “program” is used herein in a generic sense to refer to any type of computer code or set of instructions that can be employed to program a computer or other processor to implement various aspects of the present invention as discussed above. Additionally, it should be appreciated that according to one aspect of this embodiment, one or more computer programs that, when executed, perform methods of the present invention need not reside on a single computer or processor, but may be distributed in a modular fashion amongst a number of different computers or processors to implement various aspects of the present invention.

Various aspects of the present invention may be used alone, in combination, or in a variety of arrangements not specifically discussed in the embodiments described in the foregoing, and the aspects of the present invention described herein are not limited in their application to the details and arrangements of components set forth in the foregoing description or illustrated in the drawings. The aspects of the invention are capable of other embodiments and of being practiced or of being carried out in various ways. Various aspects of the present invention may be implemented on any type of computer, computer system, or any type of network connecting one or more computers. Accordingly, the foregoing description and drawings are by way of example only.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Also, the phraseology and terminology used herein is for the purpose of description and should not be regarded as limiting. The use of “including,” “comprising,” or “having,” “containing”, “involving”, and variations thereof herein, is meant to encompass the items listed thereafter and equivalents thereof as well as additional items.

What is claimed is:

1. A medical surveillance system for monitoring the safety of at least one medical entity, the medical surveillance system comprising:
at least one database to store information related to the at least one medical entity, the at least one database being periodically updated with current information about the at least one medical entity; and
at least one controller coupled to the database, the at least one controller configured to perform at least one statistical operation on at least a portion of the information stored in the at least one database, the at least one statistical operation being performed periodically so as to generate at least one continuous outcome, the controller configured to trigger an alert when the at least one continuous outcome indicates the occurrence of at least one adverse event associated with the at least one medical entity.

2. The medical surveillance system of claim 1, wherein the at least one statistical operation is performed each time the controller determines that the at least one database has been updated with current information.

3. The medical surveillance system of claim 1, wherein the medical entity includes a medical device.

4. The medical surveillance system of claim 3, wherein the at least one continuous outcome is indicative of a failure rate of the medical device, and wherein the controller triggers an alert when the at least one continuous outcome indicates that the failure rate has exceeded a first threshold.

5. The medical surveillance system of claim 1, wherein the at least one statistical operation includes using Bayesian updated statistical (BUS) analysis to generate a first continuous outcome.

6. The medical surveillance system of claim 5, wherein the at least one statistical operation includes using statistical process control (SPC) to generate a second continuous outcome and linear regression (LR) model to generate a third continuous outcome.

7. The medical surveillance system of claim 6, wherein the controller is configured to trigger the alert if any of the first continuous outcome, the second continuous outcome and the third continuous outcome indicate the occurrence of the at least one adverse event.

8. The medical surveillance system of claim 6, wherein the controller is configured to trigger the alert based on a weighted average of the first continuous outcome, the second continuous outcome and the third continuous outcome.

9. The medical surveillance system of claim 3, wherein the at least one database includes a database storing federally mandated post-market medical device safety information.

10. The medical surveillance system of claim 1, wherein the medical entity includes at least one medical procedure.

11. The medical surveillance system of claim 1, wherein the medical entity includes at least one drug.

12. The medical surveillance system of claim 1, wherein the at least one statistical operation includes a plurality of statistical operations generating a respective plurality of continuous outcomes, and wherein the alert includes a meta-alert indicating an occurrence of at least one adverse event, the meta-alert being triggered based on an analysis of the plurality of continuous outcomes.

13. The medical surveillance system of claim 12, wherein each of the plurality of statistical operations generates an alert when the respective continuous outcome is outside a predetermined threshold, and wherein the meta-alert is triggered if a respective predetermined number of the alerts are generated over an observation interval.

14. The medical surveillance system of claim 13, wherein the plurality of statistical operations include using at least two of a Bayesian updated statistical (BUS) model, statistical process control (SPC), and linear regression (LR).

15. The medical surveillance system of claim 1, wherein the information related to the at least one medical entity includes information about results of the at least one medical entity being applied to respective subjects, the subjects being categorized by a risk stratification comprised of a plurality of risk strata associated with characteristics of the subjects, and wherein the at least one statistical operation generates at least one continuous outcome for each of the plurality of risk strata.

16. The medical surveillance system of claim 15, wherein each of the plurality of risk strata is defined by at least one category.

17. The medical surveillance system of claim 16, wherein the at least one category includes an age category.

18. A method of monitoring the safety of at least one medical entity, the method comprising acts of:
obtaining periodically updated information relating to the safety of the at least one medical entity;
periodically performing at least one statistical operation on at least a portion of the information to generate at least one continuous outcome; and
generating an alert when the at least one continuous outcome indicates the occurrence of at least one adverse event associated with the at least one medical entity.

19. The method of claim 18, wherein periodically performing the at least one statistical operation includes performing at least one statistical operation each time updated information is obtained.

20. The method of claim 19, wherein the medical entity includes a medical device.

21. The method of claim 20, wherein the at least one continuous outcome is indicative of a failure rate of the medical device, and wherein the act of generating the alert includes an act of generating an alert when the at least one continuous outcome indicates that the failure rate has exceeded a first threshold.

22. The method of claim 18, wherein the act of performing the at least one statistical operation includes an act of performing a Bayesian updated statistical (BUS) analysis to generate a first continuous outcome.

23. The method of claim 22, wherein the act of performing at least one statistical operation includes an act of performing statistical process control (SPC) to generate a second continuous outcome and performing linear regression (LR) to generate a third continuous outcome.

24. The method of claim 23, wherein the act of generating the alert includes an act of generating an alert if any of the first continuous outcome, the second continuous outcome and the third continuous outcome indicate the occurrence of the at least one adverse event.

25. The method of claim 23, wherein the act of generating the alert includes an act of generating an alert based on a weighted average of the first continuous outcome, the second continuous outcome and the third continuous outcome.

26. The method of claim 20, wherein the information includes federally mandated post-market medical device safety information.

27. The method of claim 18, wherein the medical entity includes at least one medical procedure.

28. The method of claim 18, wherein the medical entity includes at least one drug.

29. The method of claim 18, wherein the at least one statistical operation includes a plurality of statistical operations generating a respective plurality of continuous outcomes, and wherein the alert includes a meta-alert indicating an occurrence of at least one adverse event, the meta-alert being triggered based on an analysis of the plurality of continuous outcomes.

30. The method of claim 29, wherein each of the plurality of statistical operations generates an alert when the respective continuous outcome is outside a predetermined threshold, and wherein the meta-alert is triggered if a respective predetermined number of the alerts are generated over an observation interval.

31. The method of claim 30, wherein the plurality of statistical operations include using at least two of a Bayesian updated statistical (BUS) analysis, statistical process control (SPC), and linear regression (LR).

32. The method of claim 18, wherein the information related to the at least one medical entity includes information about results of the at least one medical entity being applied to respective subjects, the subjects being categorized by a risk stratification comprised of a plurality of risk strata associated with characteristics of the subjects, and wherein the at least one statistical operation generates at least one continuous outcome for each of the plurality of risk strata.

33. The method of claim 32, wherein each of the plurality of risk strata is defined by at least one category.

34. The method of claim 33, wherein the at least one category includes an age category.

35. A computer readable medium encoded with a program for execution on at least one processor, the program, when executed on the at least one processor, performing a method of monitoring the safety of at least one medical entity, the method comprising acts of:
obtaining periodically updated information relating to the safety of the at least one medical entity;
periodically performing at least one statistical operation on at least a portion of the information to generate at least one continuous outcome; and
generating an alert when the at least one continuous outcome indicates the occurrence of at least one adverse event associated with the at least one medical entity.

36. The computer readable medium of claim 35, wherein periodically performing the at least one statistical operation includes performing at least one statistical operation each time updated information is obtained.

37. The computer readable medium of claim 36, wherein the medical entity includes a medical device.

38. The computer readable medium of claim 37, wherein the at least one continuous outcome is indicative of a failure rate of the medical device, and wherein the act of generating the alert includes an act of generating an alert when the at least one continuous outcome indicates that the failure rate has exceeded a first threshold.

39. The computer readable medium of claim 35, wherein the act of performing the at least one statistical operation includes an act of performing a Bayesian updated statistical (BUS) analysis to generate a first continuous outcome.

40. The computer readable medium of claim 39, wherein the act of performing at least one statistical operation includes an act of performing statistical process control (SPC) analysis to generate a second continuous outcome and linear regression (LR) analysis to generate a third continuous outcome.

41. The computer readable medium of claim 40, wherein the act of generating the alert includes an act of generating an alert if any of the first continuous outcome, the second continuous outcome and the third continuous outcome indicates the occurrence of the at least one adverse event.

42. The computer readable medium of claim 40, wherein the act of generating the alert includes an act of generating an alert based on a weighted average of the first continuous outcome, the second continuous outcome and the third continuous outcome.

43. The computer readable medium of claim 37, wherein the information includes federally mandated post-market medical device safety information.

44. The computer readable medium of claim 35, wherein the medical entity includes at least one medical procedure.

45. The computer readable medium of claim 35, wherein the medical entity includes at least one drug.

46. The computer readable medium of claim 35, wherein the at least one statistical operation includes a plurality of statistical operations generating a respective plurality of continuous outcomes, and wherein the alert includes a meta-alert indicating an occurrence of at least one adverse event, the meta-alert being triggered based on an analysis of the plurality of continuous outcomes.

47. The computer readable medium of claim 46, wherein each of the plurality of statistical operations generates an alert when the respective continuous outcome is outside a predetermined threshold, and wherein the meta-alert is triggered if a respective predetermined number of the alerts are generated over an observation interval.

48. The computer readable medium of claim 47, wherein the plurality of statistical operations include using at least two of a Bayesian updated statistical (BUS) analysis, statistical process control (SPC), and linear regression (LR).

49. The computer readable medium of claim 35, wherein the information related to the at least one medical entity includes information about results of the at least one medical entity being applied to respective subjects, the subjects being categorized by a risk stratification comprised of a plurality of risk strata associated with characteristics of the subjects, and wherein the at least one statistical operation is generates at least one continuous outcome for each of the plurality of risk strata.

50. The computer readable medium of claim 49, wherein each of the plurality of risk strata is defined by at least one category.

51. The computer readable medium of claim 50, wherein the at least one category includes an age category.
